(19) United States
(12) Patent Application Publication
(10) Pub. No.: US 2007/0066952 A1
(43) Pub. Date: Mar. 22, 2007

(54) Disposable absorbent article having deployable belt strips

(76) Inventors: Gary Dean LaVon, Liberty Township, OH (US); Kenneth Michael Hamall, West Chester, OH (US); Theodora Beck, Colerain Township, OH (US); Michael Patrick Hayden, Mason, OH (US); Susan Joy Ludwig, West Chester, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/231,511

(22) Filed: Sep. 21, 2005

(51) Int. Cl. A61F 13/15

(52) U.S. Cl. 604/392

(57) ABSTRACT

A disposable absorbent article having at least one deployable belt strip. The belt strip has a fixed end portion disposed in one of the waist regions and opposing first and second edges connecting the fixed end portion and an opposing free end portion. The belt strip is attached in the fixed end portion and is deployed by being folded laterally outward such that the first edge extends laterally outward from one end point of a diagonal fold line and the second edge extends laterally outward from the opposing end point of the diagonal fold line. The belt strip may be tied to another belt strip or may be fastened to the waist region of the article or to another belt strip.

### FIELD OF THE INVENTION

This invention relates to disposable absorbent articles such as disposable diapers and other articles intended for use on incontinent persons.

### BACKGROUND OF THE INVENTION

Disposable absorbent articles are designed to absorb and contain bodily waste in order to prevent soiling of the body and clothing of the wearer, as well as bedding or other objects with which the wearer comes into contact.

As the usage of disposable absorbent articles has expanded, their complexity has increased with the incorporation of additional features serving to enhance their performance and appearance. Among these are often complex waist closure components for application onto the body of a wearer. The costs of the materials and the costs of the manufacturing processes have also increased in conjunction with the increase in complexity. As a result, the prices at which these articles are sold have risen to levels that many potential purchasers around the world cannot afford to pay. Thus, a need exists for a disposable absorbent article having simple and cost-effective waist closure means.

### SUMMARY OF THE INVENTION

The present invention is a disposable absorbent article having at least one deployable belt strip. The belt strip has a fixed end portion disposed in one of the waist regions and opposing first and second edges connecting the fixed end portion and an opposing free end portion. The belt strip is attached in the fixed end portion and is deployed by being folded laterally outward such that the first edge extends laterally outward from one end point of a diagonal fold line and the second edge extends laterally outward from the opposing end point of the diagonal fold line. The belt strip may be tied to another belt strip or may be fastened to the waist region of the article or to another belt strip.

### BRIEF DESCRIPTION OF THE DRAWINGS

In the accompanying drawing figures, like reference numerals identify structurally corresponding elements, which may or may not be identical in the several exemplary embodiments that are depicted. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description.

In the drawing figures and in the written description, lowercase letters appended to reference numerals indicate generally symmetric elements, e.g., left and right symmetric elements may be respectively identified by the reference numerals 1a and 1b. A reference numeral without an appended lowercase letter identifies all of the elements to which that particular reference numeral applies, e.g., the same elements as a group may be designated 1.

FIG. 1 is a plan view of an exemplary disposable absorbent article in the form of a disposable diaper 20 in which the interior portion of the diaper is shown facing the viewer.

FIG. 2 is another interior plan view of the diaper 20 of FIG. 1 in which the belt strips 500 have been deployed by being folded laterally outward.

FIG. 3 is a perspective view of the diaper 20 of FIG. 2.

FIG. 4, FIG. 5, and FIG. 6 are respectively simplified side, front, and back elevation views of the diaper 20 of FIG. 1 being worn about the lower torso of a wearer.

FIG. 7, FIG. 8, and FIG. 9 are plan views of portions of exemplary diapers 20 showing alternative attachment patterns 508.

FIG. 10 is an exterior plan view of another exemplary disposable diaper 20.

FIG. 11 is another exterior plan view of the diaper 20 of FIG. 10 in which the belt strips 500 have been deployed by being folded laterally outward.

FIG. 12 is a perspective view of the diaper 20 of FIG. 11.

FIG. 13, FIG. 14, and FIG. 15 are respectively simplified side, front, and back elevation views of the diaper 20 of FIG. 10 being worn about the lower torso of a wearer.

FIG. 16, FIG. 17, and FIG. 18 are plan views of portions of exemplary diapers 20.

FIG. 19 is an exterior plan view of another exemplary disposable diaper 20.

FIG. 20 is an exterior plan view of another exemplary disposable diaper 20.

FIG. 21 is a perspective view of the diaper 20 of FIG. 20 showing the belt strips 500 deployed.

FIG. 22 and FIG. 23 are respectively simplified side and front elevation views of the diaper 20 of FIG. 20 being worn about the lower torso of a wearer.

FIG. 24 is an exterior plan view of another exemplary disposable diaper 20.

FIG. 25 is a perspective view of the diaper 20 of FIG. 24 showing the belt strips 500 deployed.

FIG. 26 and FIG. 27 are respectively simplified side and front elevation views of the diaper 20 of FIG. 24 being worn about the lower torso of a wearer.

FIG. 28 is an exterior plan view of another exemplary disposable diaper 20.

FIG. 29 is a perspective view of the diaper 20 of FIG. 28 showing the belt strips 500 deployed.

FIG. 30 is a simplified side elevation view of the diaper 20 of FIG. 28 being worn about the lower torso of a wearer.

FIG. 31 is an exterior plan view of another exemplary disposable diaper 20.

FIG. 32, FIG. 33, and FIG. 34 are respectively simplified side, front, and back elevation views of the diaper 20 of FIG. 31 being worn about the lower torso of a wearer.

FIG. 35 is an exterior plan view of another exemplary disposable diaper 20.

FIG. 36 is a perspective view of an alternative embodiment of a diaper 20.

FIG. 37 is a perspective view of another alternative embodiment of a diaper 20.

FIG. 38 is an interior plan view of another exemplary disposable diaper 20.

FIG. 39 is an exterior plan view of the diaper 20 of FIG. 38.

FIG. 40, FIG. 41, and FIG. 42 are section views of the diaper 20 of FIG. 38 and FIG. 39 taken at the respective section lines 40-40, 41-41, and 42-42. In these section views, the interior portion of the diaper 20 is shown facing upward.

FIG. 43, FIG. 44, and FIG. 45 are section views of an alternative embodiment of a diaper 20 taken at section lines corresponding to the respective section lines 40-40, 41-41, and 42-42. In these section views, the interior portion of the diaper 20 is shown facing upward.

FIG. 46, FIG. 47, and FIG. 48 are section views of an alternative embodiment of a diaper 20 taken at section lines corresponding to the respective section lines 40-40, 41-41, and 42-42. In these section views, the interior portion of the diaper 20 is shown facing upward.

FIG. 49, FIG. 50, and FIG. 51 are section views of an alternative embodiment of a diaper 20 taken at section lines corresponding to the respective section lines 40-40, 41-41, and 42-42. In these section views, the interior portion of the diaper 20 is shown facing upward.

FIG. 52, FIG. 53, and FIG. 54 are section views of an alternative embodiment of a diaper 20 taken at section lines corresponding to the respective section lines 40-40, 41-41, and 42-42. In these section views, the interior portion of the diaper 20 is shown facing upward.

FIG. 55 is an interior plan view of another exemplary disposable diaper 20.

FIG. 56 is an exterior plan view of the diaper 20 of FIG. 55.

FIG. 57, FIG. 58, and FIG. 59 are section views of the diaper 20 of FIG. 38 and FIG. 39 taken at the respective section lines 57-57, 58-58, and 59-59. In these section views, the interior portion of the diaper 20 is shown facing upward.

FIG. 60, FIG. 61, and FIG. 62 are section views of an alternative embodiment of a diaper 20 taken at section lines corresponding to the respective section lines 57-57, 58-58, and 59-59. In these section views, the interior portion of the diaper 20 is shown facing upward.

FIG. 63, FIG. 64, and FIG. 65 are section views of an alternative embodiment of a diaper 20 taken at section lines corresponding to the respective section lines 57-57, 58-58, and 59-59. In these section views, the interior portion of the diaper 20 is shown facing upward.

FIG. 66, FIG. 67, and FIG. 68 are section views of an alternative embodiment of a diaper 20 taken at section lines corresponding to the respective section lines 57-57, 58-58, and 59-59. In these section views, the interior portion of the diaper 20 is shown facing upward.

FIG. 69, FIG. 70, and FIG. 71 are section views of an alternative embodiment of a diaper 20 taken at section lines corresponding to the respective section lines 57-57, 58-58, and 59-59. In these section views, the interior portion of the diaper 20 is shown facing upward.

FIG. 72, FIG. 73, and FIG. 74 are section views of an alternative embodiment of a diaper 20 taken at section lines corresponding to the respective section lines 57-57, 58-58, and 59-59. In these section views, the interior portion of the diaper 20 is shown facing upward.

FIG. 75, FIG. 76, and FIG. 77 are section views of an alternative embodiment of a diaper 20 taken at section lines corresponding to the respective section lines 57-57, 58-58, and 59-59. In these section views, the interior portion of the diaper 20 is shown facing upward.

FIG. 78, FIG. 79, and FIG. 80 are section views of an alternative embodiment of a diaper 20 taken at section lines corresponding to the respective section lines 57-57, 58-58, and 59-59. In these section views, the interior portion of the diaper 20 is shown facing upward.

FIG. 81, FIG. 82, and FIG. 83 are section views of an alternative embodiment of a diaper 20 taken at section lines corresponding to the respective section lines 57-57, 58-58, and 59-59. In these section views, the interior portion of the diaper 20 is shown facing upward.

FIG. 84, FIG. 85, and FIG. 86 are section views of an alternative embodiment of a diaper 20 taken at section lines corresponding to the respective section lines 57-57, 58-58, and 59-59. In these section views, the interior portion of the diaper 20 is shown facing upward.

FIG. 87, FIG. 88, and FIG. 89 are section views of an alternative embodiment of a diaper 20 taken at section lines corresponding to the respective section lines 57-57, 58-58, and 59-59. In these section views, the interior portion of the diaper 20 is shown facing upward.

### DETAILED DESCRIPTION OF THE INVENTION

In this description, the following terms have the following meanings:

The term “absorbent article” refers to a device that absorbs and contains liquid, and more specifically, refers to a device that is placed against or in proximity to the body of a wearer to absorb and contain the various exudates discharged from the body.

The term “diaper” refers to an absorbent article that is generally worn by infants and incontinent persons about the lower torso so as to encircle the waist and the legs of the wearer and that is specifically adapted to receive and contain urinary and fecal waste.

The term “disposable” refers to the nature of absorbent articles that generally are not intended to be laundered or otherwise restored or reused as an absorbent article, i.e., they are intended to be discarded after a single use and, preferably, to be recycled, composted or otherwise disposed of in an environmentally compatible manner. In this description, a disposable diaper is described as being representative of an exemplary disposable absorbent article.

The term “deploy” in all its forms refers to the manipulation of the disclosed belt strips from their initial configuration to a configuration in which they can be used to at least partially encircle the waist of a wearer of the article on which they are provided.

The term “longitudinal” refers to a direction running from a waist edge to an opposing waist edge of the article and generally parallel to the maximum linear dimension of the article.

The term “lateral” refers to a direction running from a side edge to an opposing side edge of the article and generally at a right angle to the longitudinal direction.

The term “diagonal” refers to an orientation of a line extending obliquely relative to the longitudinal and lateral directions, i.e., neither perpendicular nor parallel to either of the longitudinal or lateral directions.

The term “disposed” refers to an element being attached and positioned in a particular place or position in a unitary structure with other elements.

The term “attached” refers to elements being connected or united by fastening, adhering, bonding, etc. by any method suitable for the elements being attached together and their constituent materials. Many suitable methods for attaching elements together are well-known, including adhesive bonding, pressure bonding, thermal bonding, mechanical fastening, etc. Such attachment methods may be used to attach elements together over a particular area either continuously or intermittently.

The term “cohesive” refers to the property of a material that, once set, sticks to itself but does not to any significant degree stick to other materials.

The terms “water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water to pass through its thickness in the absence of a forcing pressure. Conversely, the term “water-impermeable” refers to a layer or a layered structure through the thickness of which liquid water cannot pass in the absence of a forcing pressure. A layer or a layered structure that is water-impermeable according to this definition may be permeable to water vapor, i.e., may be “water vapor-permeable”. Such a water vapor-permeable layer or layered structure is commonly known in the art as “breathable”. As is well known in the art, a common method for measuring the permeability to water of the materials typically used in absorbent articles is a hydrostatic pressure test, also called a hydrostatic head test or simply a “hydrohead” test. Suitable well known compendial methods for hydrohead testing are approved by INDA (formerly the International Nonwovens and Disposables Association, now The Association of the Nonwoven Fabrics Industry) and EDANA (European Disposables And Nonwovens Association).

The terms “proximal” and “distal” refer respectively to the location of an element relatively near to or far from the center of a structure, e.g., the laterally proximal edge of a longitudinally extending element is located nearer to the longitudinal axis than the laterally distal edge of the same element is located relative to the same longitudinal axis. When used to describe relative locations with respect to the axes, synonyms include “inboard” and “outboard”, respectively.

The terms “interior” and “exterior” refer respectively to the location of an element that is intended to be placed against or toward the body of a wearer when an absorbent article is worn and the location of an element that is intended to be placed against or toward any clothing that is worn over the absorbent article. Synonyms for “interior” and “exterior” include, respectively, “inner” and “outer”, as well as “inside” and “outside”. Also, when the absorbent article is oriented such that its interior faces upward, e.g., when it is laid out in preparation for setting the wearer on top of it, synonyms include “upper” and “lower”, “above” and “below”, “over” and “under”, and “top” and “bottom”, respectively.

As can be seen in the drawing figures, one end portion of the exemplary diaper 20 is configured as a front waist region 36, the longitudinally opposing end portion is configured as a back waist region 38, and an intermediate portion is configured as a crotch region 37.

The basic structure of the diaper 20 includes a chassis 100, which has a front edge 136, a back edge 138, laterally opposing side edges 137, an interior surface 102, and an exterior surface 104. A longitudinal axis 42 extends through the midpoints of the front edge 136 and the back edge 138 and a lateral axis 44 extends through the midpoints of the side edges 137.

The basic structure of the diaper 20 also includes an absorbent assembly 200, which is attached to the chassis 100. The absorbent assembly 200 absorbs and retains liquid bodily waste materials. Suitable well-known absorbent materials for the absorbent assembly include cellulose fibers in the form of comminuted wood pulp, which is commonly known as “airfelt”, layers or sheets of natural or synthetic fibrous material, superabsorbent polymer, etc. These absorbent materials may be used separately or in combination and many may be used in a discrete form, i.e., in the form of fibers, granules, particles, layers and the like. The discrete form of an absorbent material may be immobilized in pockets formed by a layer of a thermoplastic material, such as a hot melt adhesive, that intermittently contacts and adheres to a substrate, such as a covering sheet, while diverging away from the substrate at the pockets. Absorbent assemblies having such pocket structures are described in more detail in U.S. Patent Application Publications Nos. 2004/0167486 of Aug. 26, 2004 and 2004/0162536 of Aug. 19, 2004.

The basic structure of the diaper 20 also includes at least one deployable belt strip 500, as described in detail below.

When the diaper 20 is worn on the lower torso of a wearer, the front waist edge 136 and the back waist edge 138 of the chassis lie against the waist of the wearer, the side edges 137 partially or wholly encircle the legs of the wearer, the crotch region 37 is generally positioned between the legs of the wearer, and the absorbent assembly 200 extends from the front waist region 36 through the crotch region 37 to the back waist region 38.

A portion or the whole of chassis and/or the absorbent assembly and/or the belt strip may be formed of an elastically extensible material or materials. Alternatively, or in addition, a portion or the whole of chassis and/or the absorbent assembly and/or the belt strip may be made extensible to a degree greater than the inherent extensibility of the material or materials from which it is made. The additional extensibility may be desirable in order to allow the diaper 20 to conform to the body of a wearer during movement by the wearer. Additional lateral extensibility may be particularly desirable to allow the user of a diaper to extend the front waist region and/or the back waist region to encircle the waist of a wearer, i.e., to tailor the waist size and fit of a diaper to the individual wearer. Such a lateral extension of the waist region or regions may give the diaper a generally hourglass shape and may impart a tailored appearance to the diaper when it is worn. In addition, the additional extensibility may be desirable in order to minimize the cost of the diaper, because a relatively lesser amount of material is needed when the material is made extensible as described.

This additional extensibility may be provided in a variety of ways. For example, a material or materials from which the chassis and/or the absorbent assembly and/or the belt strip is/are made may be pleated by any of many known methods. Alternatively, all or a portion of the chassis and/or the absorbent assembly and/or the belt strip may be made of a formed web material or a formed laminate of web materials like those described in U.S. Pat. No. 5,518,801 issued on May 21, 1996 in the name of Chappell et al. In addition, different portions of the chassis and/or the absorbent assembly and/or the belt strip may be formed to have different ranges of extensibility and/or to be extensible to a greater or lesser degree when subjected to a given level of opposing tensile forces, i.e., to be relatively more easily or less easily extensible. Such differential extensibility may be desirable so that, for example, one or both of the waist regions may be laterally extended relatively farther or relatively more easily than the crotch region.

Unless explicitly excluded in its description or precluded by a structural characteristic unique to the particular disposition of the belt strip 500 or to the particular embodiment shown, the following description of alternatives applies to every configuration of the belt strip 500.

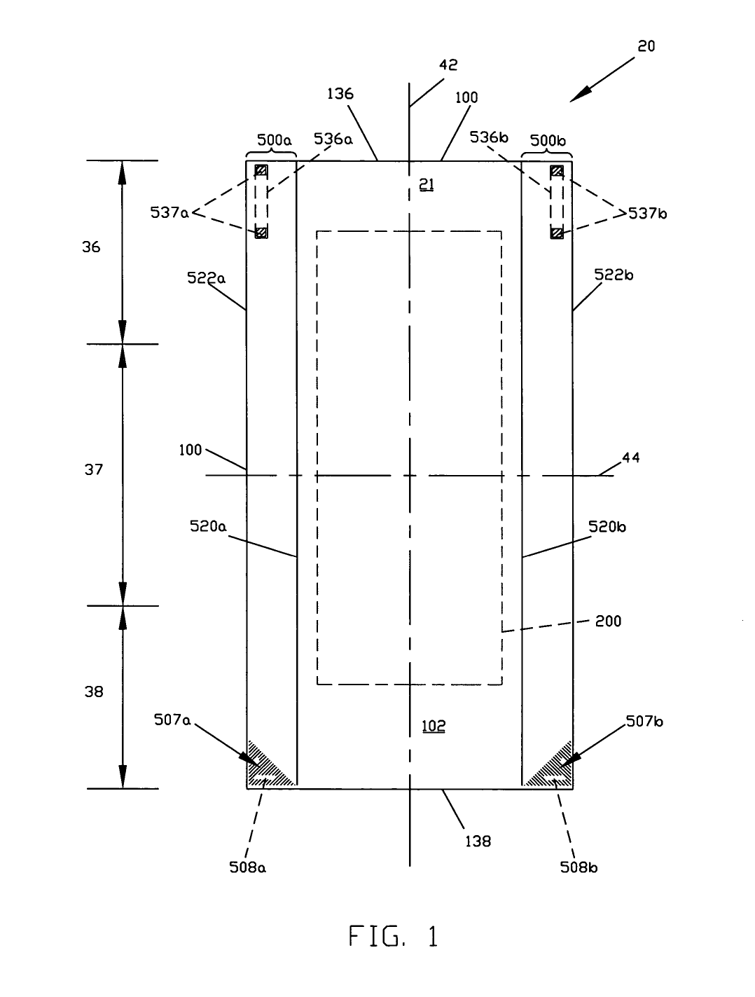

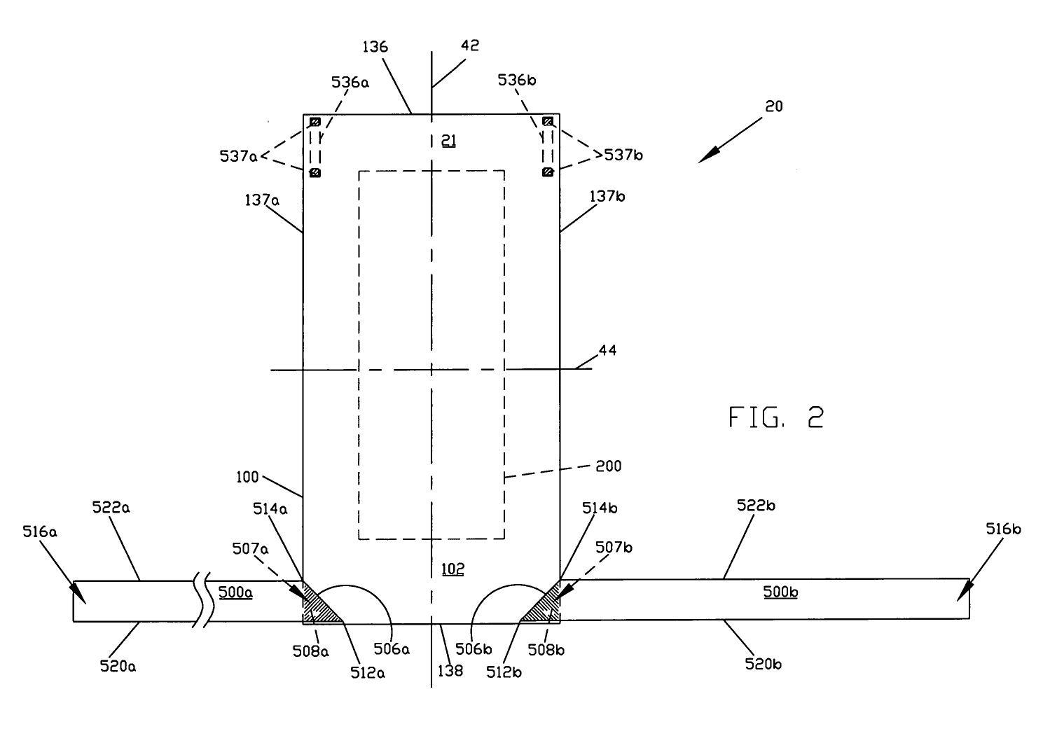

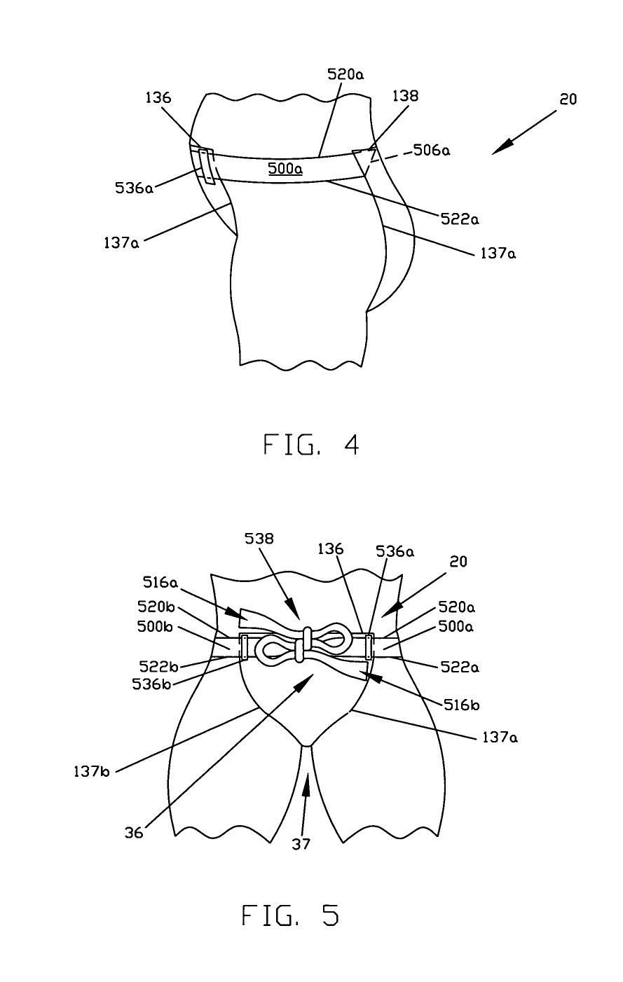

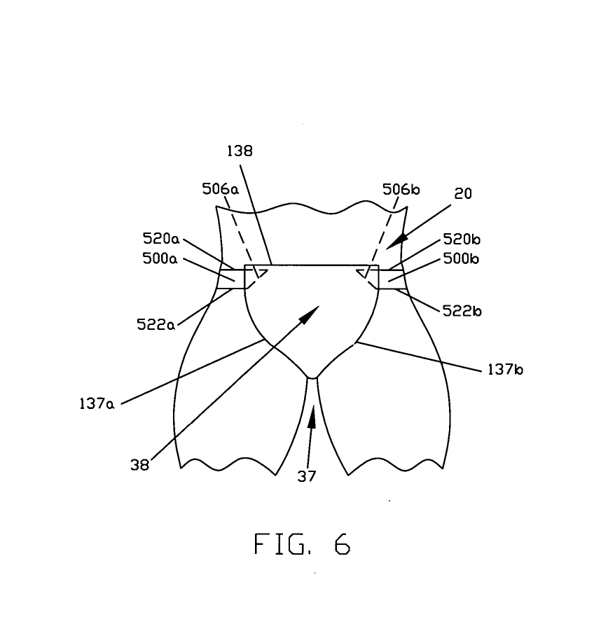

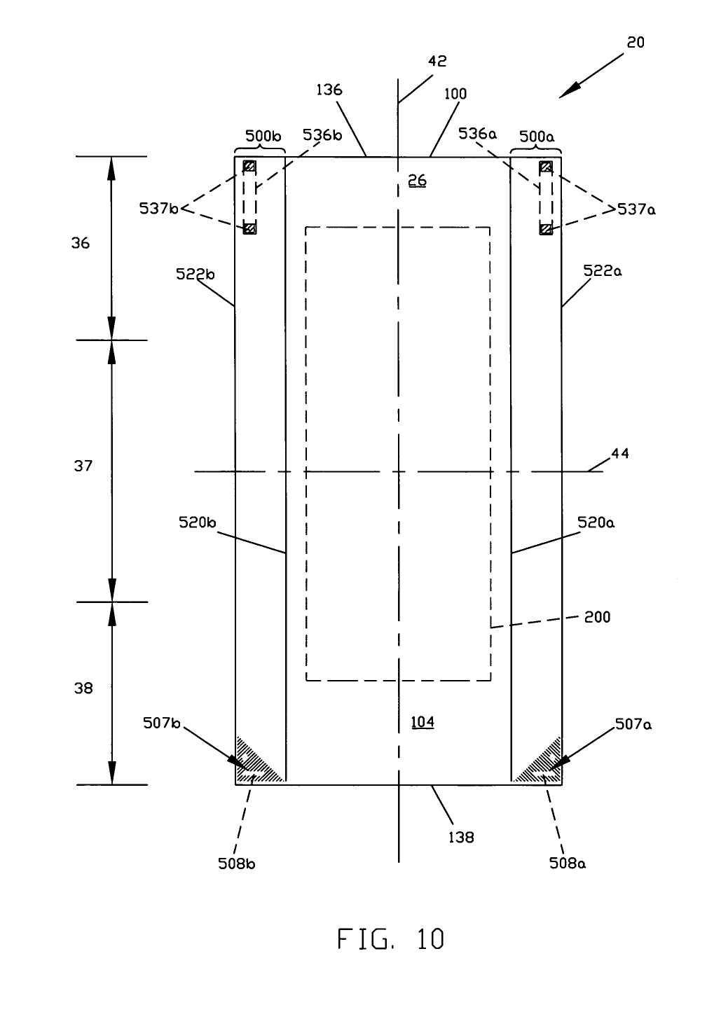

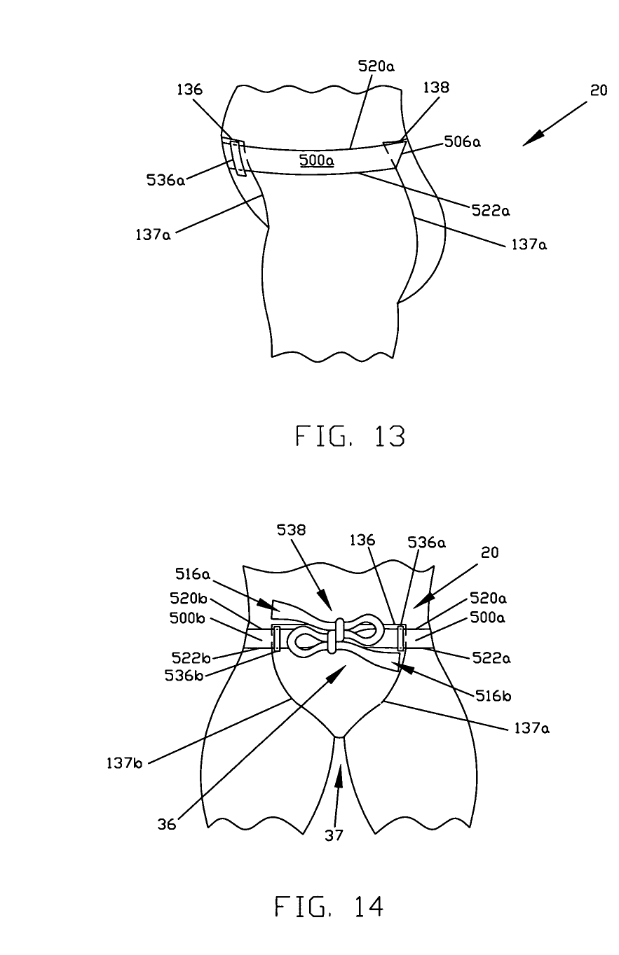

In FIG. 1 through FIG. 9, the belt strips 500 are shown disposed interiorly. Alternatively the belt strips 500 may be disposed exteriorly, as shown in FIG. 10 through FIG. 35.

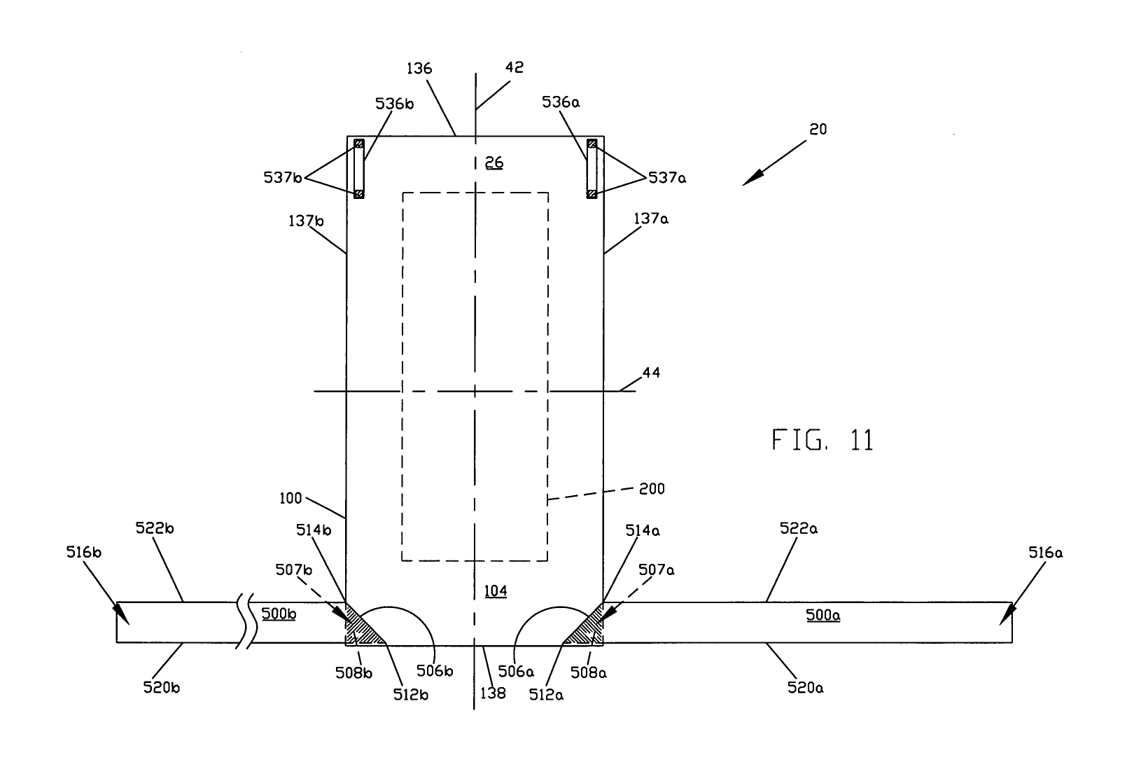

Each belt strip 500 is formed in an attached configuration as shown, for example, in FIG. 1 and in FIG. 10. The belt strip 500 is deployed for use by detaching the belt strip 500 except at its fixed end portion 507 and folding the belt strip 500 laterally outward at a diagonal fold line 506 as shown, for example, in FIG. 2 and in FIG. 11. Once deployed, each belt strip 500 is tied to another belt strip, fastened to a waist region of the diaper, and/or fastened to another belt strip in order to thereby partially or wholly encircle the waist of the wearer of the diaper 20.

In the present figures, the diagonal fold lines 506 are located adjacent to the back waist edge 138 of the diaper 20 and the belt strips 500 extend from there toward the front waist edge 136. Alternatively, the diagonal fold lines 506 may be located adjacent to the front waist edge 136 of the diaper 20, in which configuration the belt strips 500 extend toward the back waist edge 138. In general, other structural elements that are described in relation to the belt strips and whose disposition is dependent on the disposition of the belt strips may likewise be located oppositely in combination with oppositely disposed belt strips.

The belt strip 500 has a longitudinally extending first edge 520 and a laterally opposing longitudinally extending second edge 522. Each of the first and second edges is formed by either an edge of a sheet of material, a fold in a sheet of material, or a frangible separation line. The first edge 520 is located laterally proximally relative to the second edge 522 prior to the deployment of the belt strip 500 for use. When the belt strip 500 is deployed for use, the first edge 520 is positioned as the upper edge and the second edge 522 is positioned as the lower edge of the belt strip 500, i.e., the first edge 520 is disposed farther from the lateral axis 44 than the second edge 522 is disposed.

The diagonal fold line 506 has a laterally proximal end point 512 and an opposing laterally distal end point 514 located longitudinally proximally relative to the laterally proximal end point 512. In other words, the laterally distal end point 514 is located relatively closer to the lateral axis 44 of the diaper 20 than the laterally proximal end point 512 is located.

When deployed for use, the upper edge 520 of the belt strip 500 extends laterally outward from the laterally proximal end point 512 and the lower edge 522 extends laterally outward from the laterally distal end point 514. The laterally proximal end point 512 of the diagonal fold line 506 may be located at the respective waist edge or may be located below the waist edge, i.e., between the waist edge and the lateral axis 44. Thus, when the laterally proximal end point 512 is located at the waist edge, the upper edge 520 of the belt strip 500 meets the waist edge. Similarly, when the laterally proximal end point 512 is located below the waist edge, the upper edge 520 of the belt strip 500 is likewise below the waist edge.

Any portion of the chassis 100 protruding longitudinally beyond the upper edge 520 of the deployed belt strip 500 is free to fold over, either interiorly or exteriorly. Such folding over may degrade the appearance of the diaper 20 on the wearer. In addition, this folding over may negatively affect the performance of the diaper. For example, folding over into the interior may undesirably expose an exterior layer of the diaper 20, such as a plastic film, to the skin of the wearer. Conversely, folding over to the exterior may expose a wet interior layer of the diaper 20 to clothing or bedding. Therefore, it may be desirable to locate the laterally proximal end point 512 at or closely adjacent to the waist edge in order to minimize the size of any such portion of the chassis 100 protruding longitudinally beyond the upper edge 520 of the deployed belt strip 500 and thereby prevent, or at least minimize, the magnitude of any degradation in appearance and/or performance.

For the purpose of clarity in the present drawing figures, the laterally proximal end point 512 of each deployed belt strip 500 and the upper edge 520 of that deployed belt strip 500 are shown displaced slightly from the back waist edge 138 of the diaper 20, rather than being shown exactly coincident with that waist edge. This depiction is intended to represent the preference that the upper edge 520 of the deployed belt strip 500 be located either at or closely adjacent to the waist edge in order to minimize the protrusion of the chassis 100 beyond the upper edge 520, for the reason explained above. In order to locate the upper edge 520 as preferred, the distance between the laterally proximal end point 512 and the closest waist edge is less than the longitudinal distance between the laterally proximal end point 512 and the laterally distal end point 514. For example, the laterally proximal end point 512 may be located within approximately 6 mm of the closest waist edge of the diaper 20.

The diagonal fold line 506 may be oriented such that a deployed belt strip 500 extends parallel to the lateral axis 44 or at an angle with respect to the lateral axis 44. For example, a belt strip 500 formed parallel to the longitudinal axis 42 and deployed by being folded laterally outward at a fold line 506 oriented at 45 degrees to both the longitudinal axis 42 and the lateral axis 44 of the diaper 20 extends parallel to the lateral axis 44 when deployed. However, when such a longitudinally parallel formed belt strip 500 is folded at a fold line 506 oriented at an angle other than 45 degrees, the belt strip 500 extends at an angle with respect to the lateral axis 44. Similarly, a belt strip 500 formed at an angle relative to the longitudinal axis 42 and deployed by being folded laterally outward at a 45 degree diagonal fold line 506 extends at an angle with respect to the lateral axis 44. For example, in some embodiments, it may be desirable to fit the deployed belt strip 500 on the torso of a wearer along a path running from the small of the back to below the navel.

In its fixed end portion 507, the belt strip 500 is attached to another layer of the diaper 20 at both the laterally proximal end point 512 and the laterally distal end point 514 of the diagonal fold line 506 in an attachment zone 508. The attachment zone 508 may have a continuous or intermittent form, for example two points, a pattern of more than two points, a continuous area, or a pattern of discontinuous areas. Thus, the belt strip 500 may be attached either continuously or intermittently along the diagonal fold line 506 between the laterally proximal end point 512 and the laterally distal end point 514. The attachment zone 508 may be formed by any means suitable for the materials involved, including stitching, adhesive bonding, thermal bonding, stapling, and riveting, for example.

For example, as shown in FIG. 1 and in FIG. 10, the attachment zone 508 may extend longitudinally and laterally outward from the diagonal fold line 506 in directions away from both the longitudinal axis 42 and the lateral axis 44. Such a triangular attachment zone 508 may be desirable in order to strengthen and/or stabilize this area where any force exerted by a deployed belt strip 500 is transmitted to the remainder of the structure of the diaper 20.

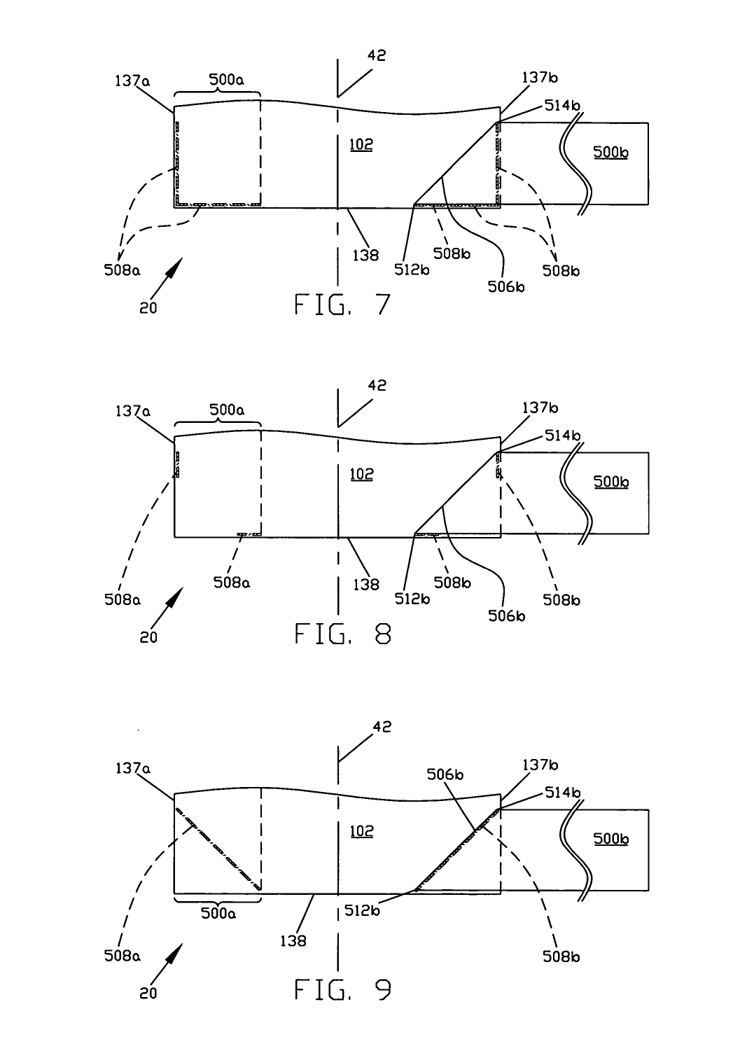

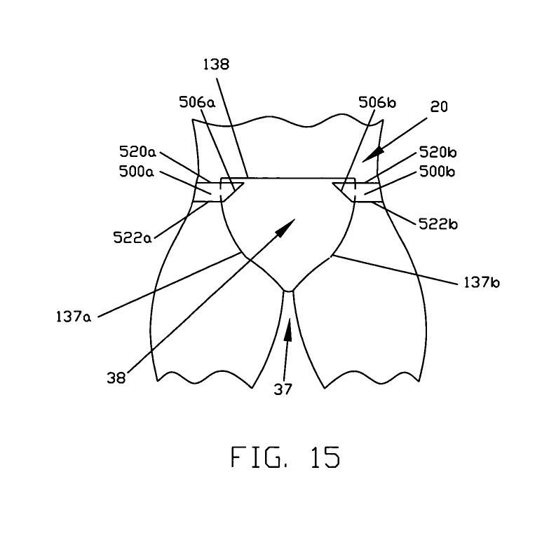

As other examples, as shown in FIG. 7 and FIG. 8 and in FIG. 16 and FIG. 17, the attachment zone 508 may extend longitudinally from the laterally distal end point 514 of the diagonal fold line 506 in a direction away from the lateral axis 44 toward or to the adjacent waist edge of the diaper 20 and laterally from the laterally proximal end point 512 of the diagonal fold line 506 in a direction in a direction away from the longitudinal axis 42 toward or to the adjacent side edge of the diaper 20, without forming a triangle. As yet another example, as shown in FIG. 9 and in FIG. 18, the attachment zone 508 may extend from the laterally proximal end point 512 toward or to the laterally distal end point 514 along the diagonal fold line 506 itself.

Because the belt strip 500 is attached at least at both ends of the diagonal fold line 506, any tension in the belt strip 500 is transmitted to the remainder of the structure of the diaper 20 over the width of the belt strip 500, rather than being concentrated at a single point. Such a distributed transmission of force may be desirable in order to minimize the possibility of marking the skin of the wearer and/or to minimize the possibility of overstressing the structure. In particular, when the belt strip 500 is attached along the entire diagonal fold line 506 or in a triangular attachment zone 508 as described above, the tensile force may be uniformly distributed across the width of the belt strip 500.

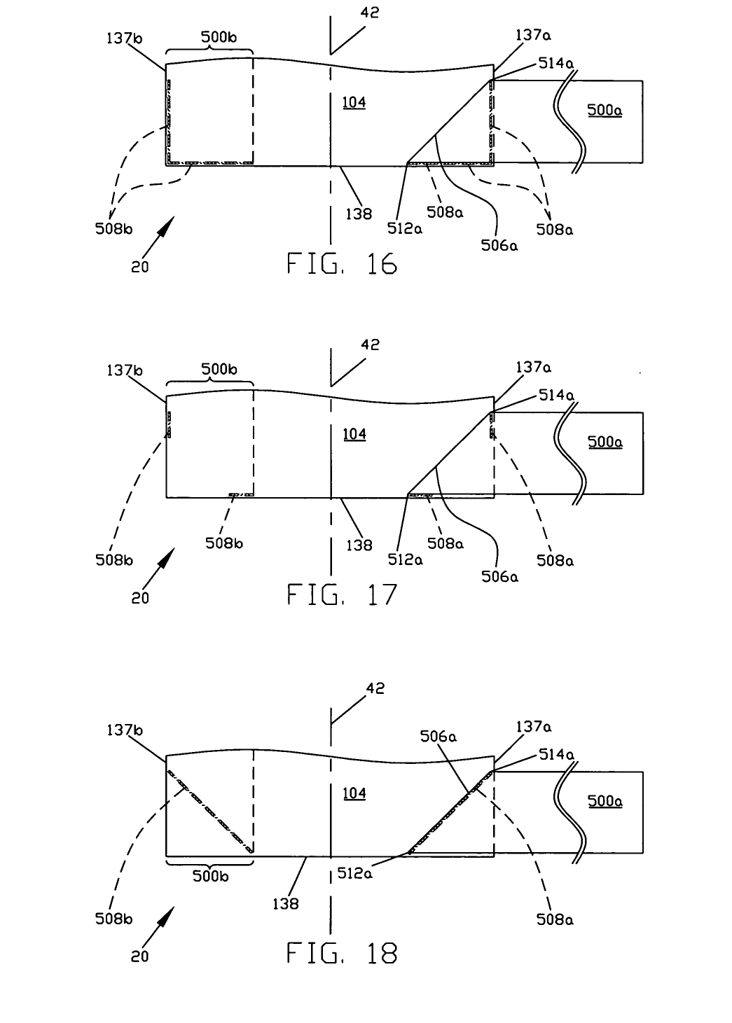

In FIG. 1 through FIG. 18, the diaper 20 has two belt strips 500 that are laterally spaced apart. Alternatively, two belt strips 500 may be laterally abutted, rather than being spaced apart. For example, in FIG. 19, the two belt strips 500 are disposed such that their respective first edges 520 extend from a common laterally proximal end point 512 of both of their diagonal fold lines 506. Thus, prior to deployment, these two belt strips 500 had a common first edge 520 extending from the common laterally proximal end point 512. In FIG. 19, the two belt strips 500 are disposed symmetrically with respect to the longitudinal axis 42 of the diaper 20. Alternatively, two laterally abutted belt strips 500 may be disposed asymmetrically with respect to the longitudinal axis 42 of the diaper 20.

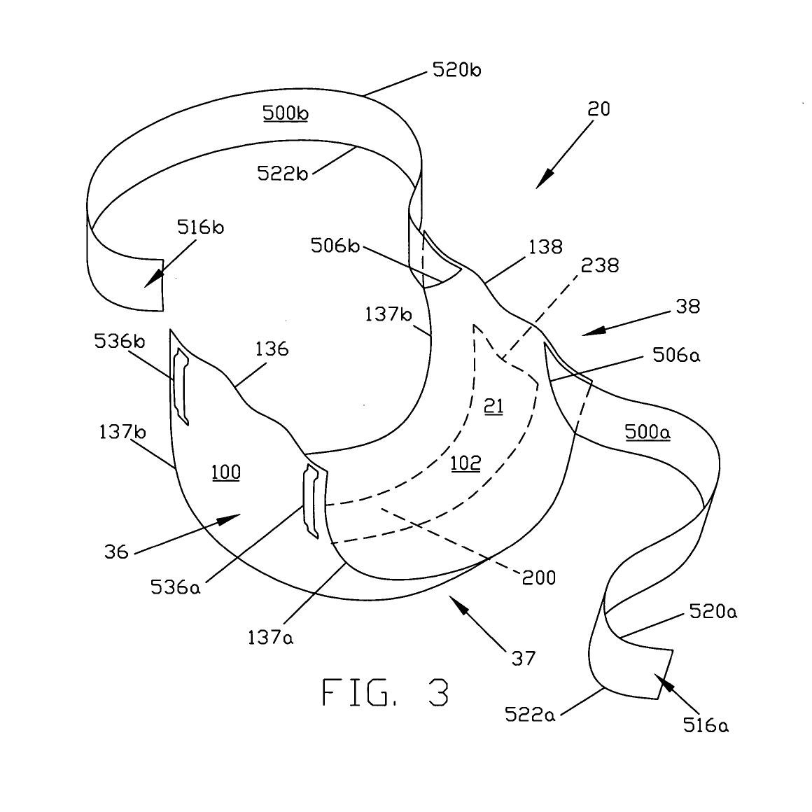

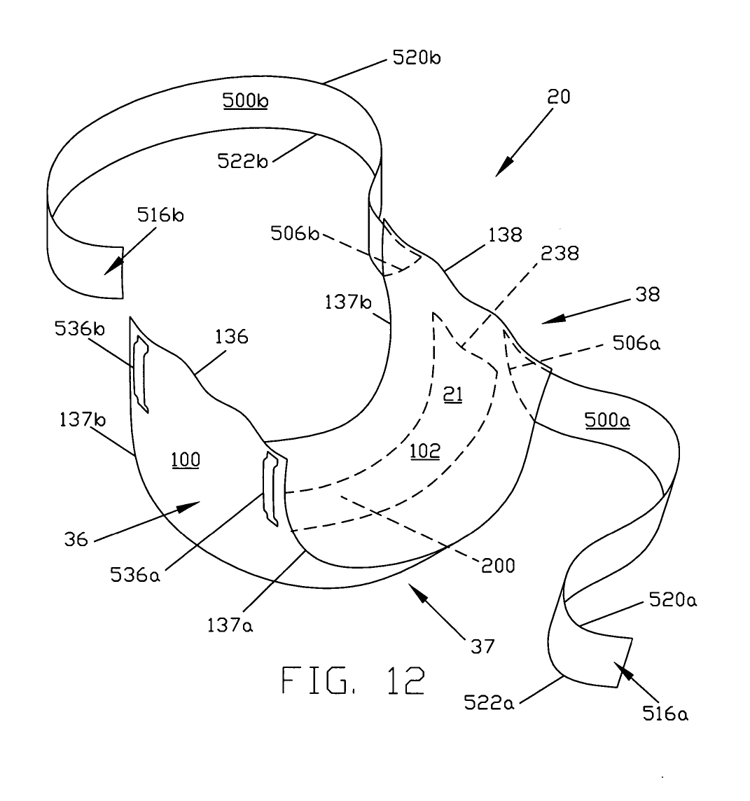

Prior to deployment for use, each belt strip 500 may extend from the laterally proximal end point 512 of the diagonal fold line 506 to the opposing waist edge. For example, in FIG. 1 and in FIG. 10, each belt strip 500 extends from its laterally proximal end point 512 located adjacent to the back waist edge 138 to the opposing front waist edge 136. When such a “full length” belt strip 500 is deployed for use, a portion of the opposing waist edge defines a free end portion 516 of the belt strip 500, as shown in FIG. 2, FIG. 3, FIG. 11, and FIG. 12.

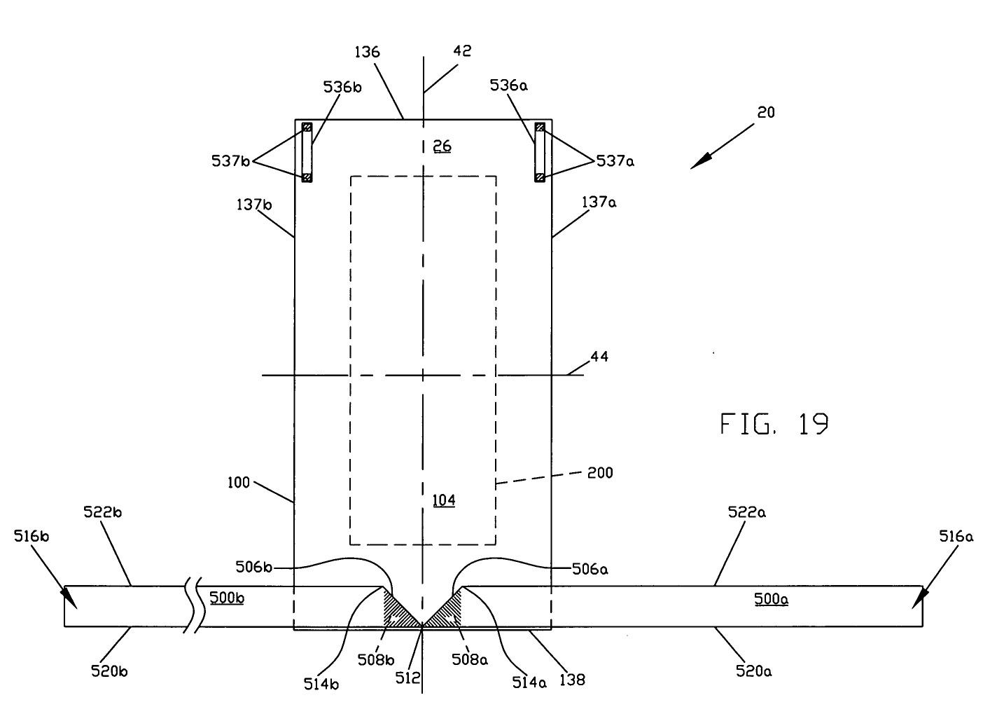

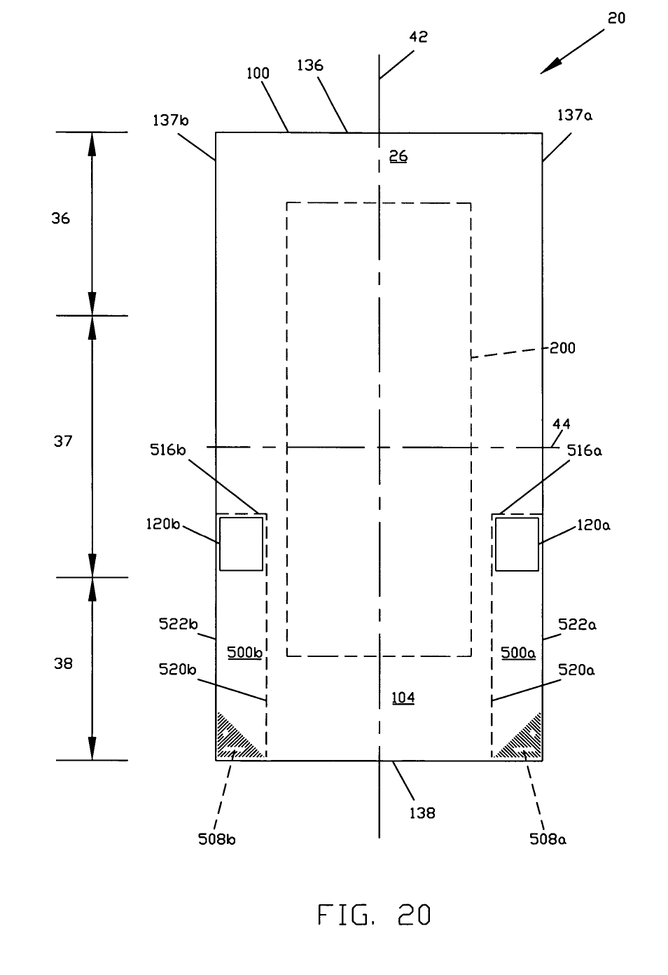

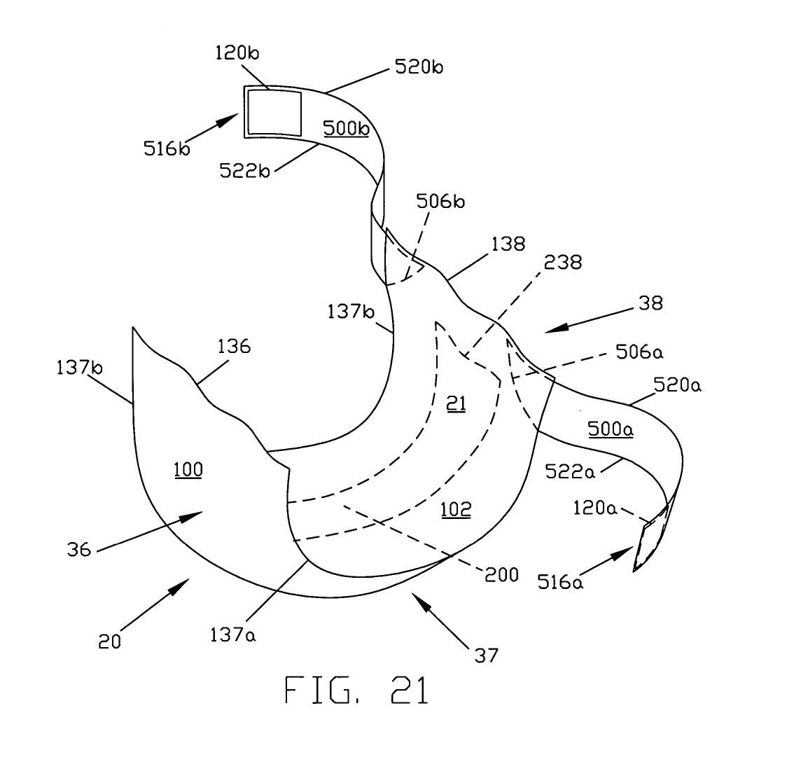

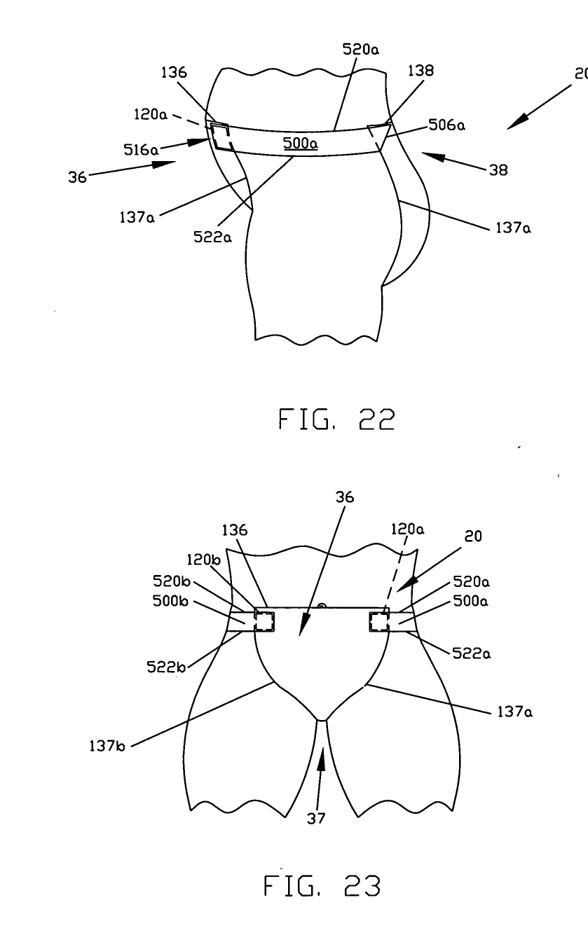

Alternatively, the belt strip 500 may extend only a part of the way between the laterally proximal end point 512 and the opposing waist edge. For example, in FIG. 20 and in FIG. 21, each belt strip 500 extends from its laterally proximal end point 512 located adjacent to the back waist edge 138 to a free end portion 516 located between the laterally proximal end point 512 and the opposing front waist edge 136. This free end 516 may be defined by a laterally extending frangible separation line. As shown in FIG. 22 and FIG. 23, when the diaper 20 is applied onto the wearer, each such partial length belt strip 500 may be used to connect the waist regions at and/or adjacent to a respective side edge of the diaper 20. Such laterally opposing partial length belt strips 500 may overlap or may end short of overlapping.

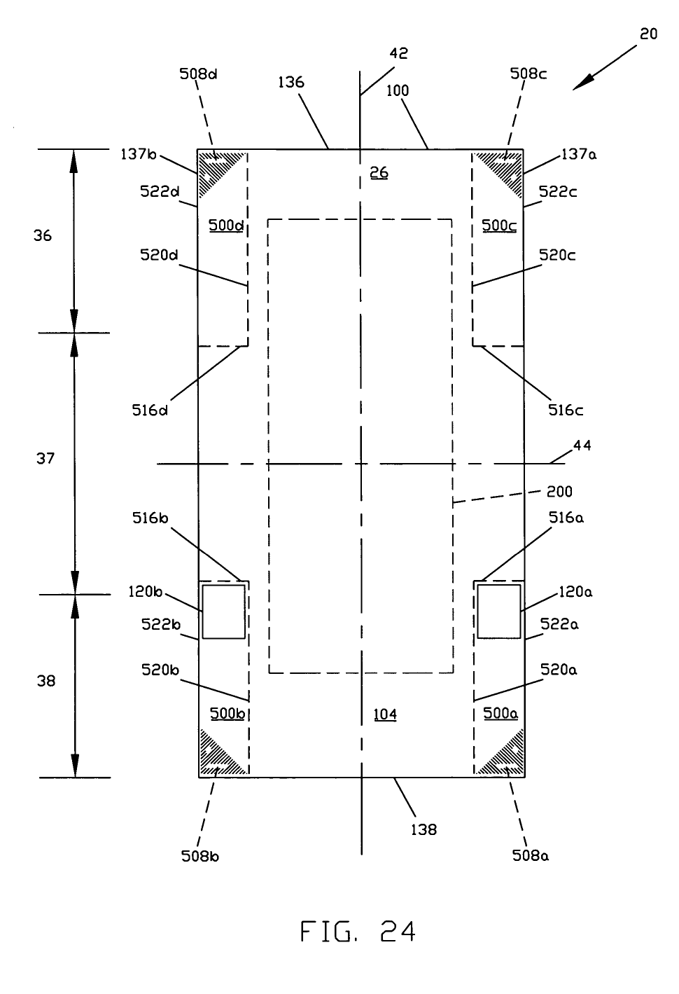

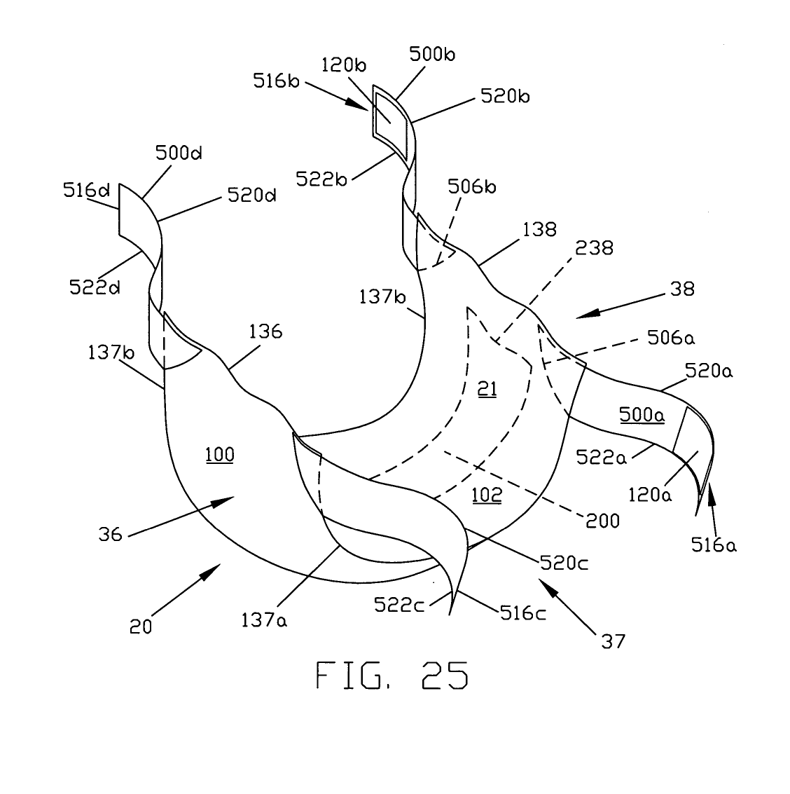

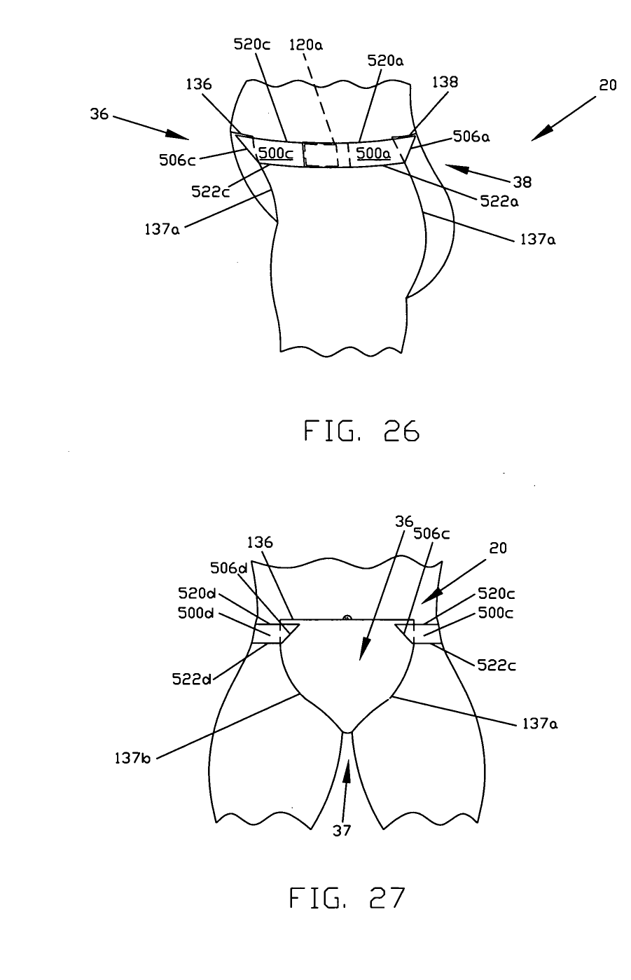

As another alternative, as shown in FIG. 24 and in FIG. 25, two longitudinally opposing partial length belt strips 500 may be formed adjacent to each side edge of the diaper 20, for a total of four belt strips 500. When the diaper 20 is applied onto the wearer, the two laterally opposing partial length belt strips 500c and 500d in the front waist region 36 and the respective laterally opposing partial length belt strips 500a and 500b in the back waist region 38 are used to connect the waist regions at and/or adjacent to the respective side edges of the diaper 20 as shown in FIG. 26 and in FIG. 27. In particular, the partial length belt strips 500a and 500c adjacent to the left side edge 137a are attached together and the partial length belt strips 500b and 500d adjacent to the right side edge 137b are attached together.

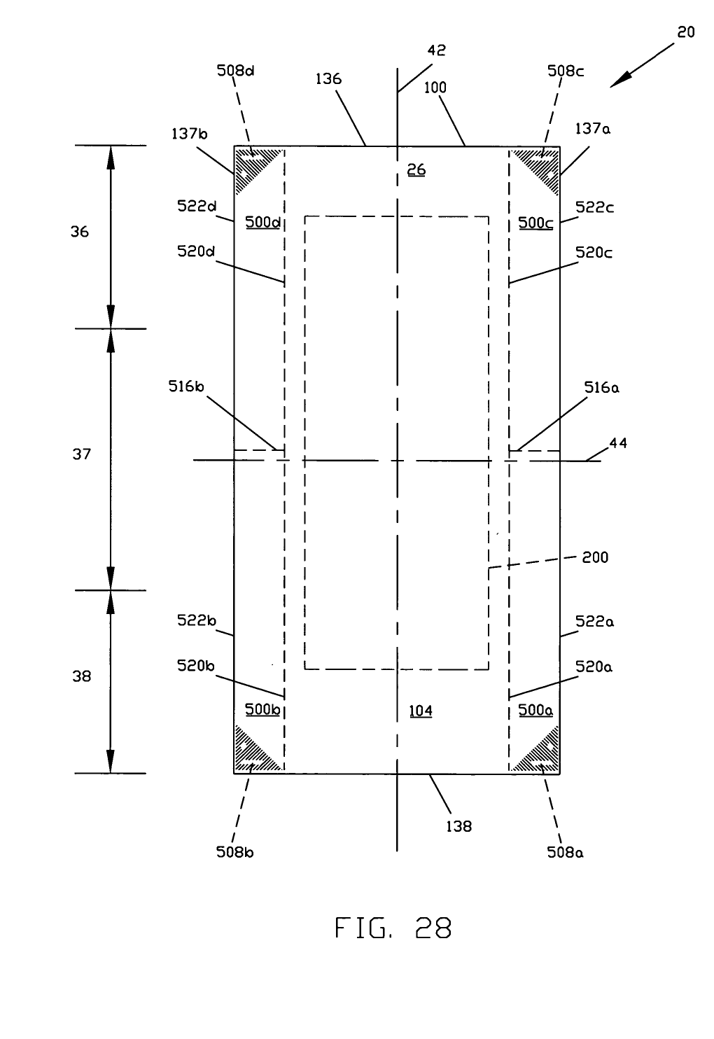

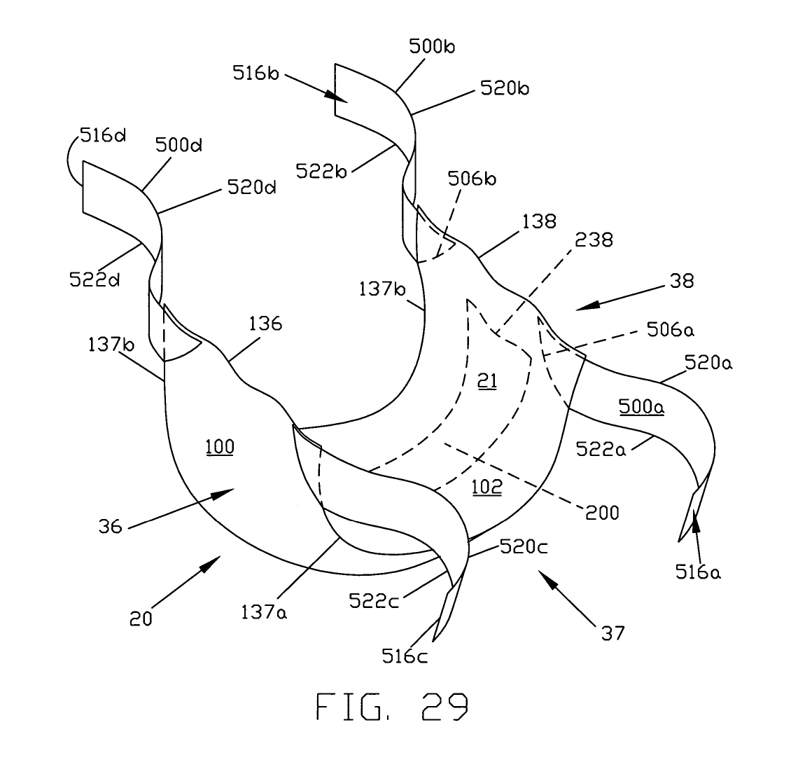

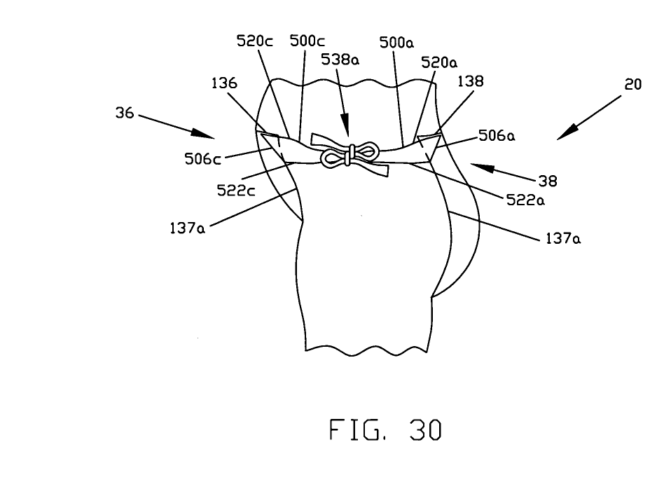

As shown in FIG. 24, the two longitudinally opposing partial-length belt strips 500 on each side may not meet. As an alternative, as shown in FIG. 28 and in FIG. 29, the two longitudinally opposing partial length belt strips 500 on each side may meet at their free end portions 516, thereby being relatively longer than in a configuration in which they do not meet, and may be long enough to be tied together, as shown in FIG. 30.

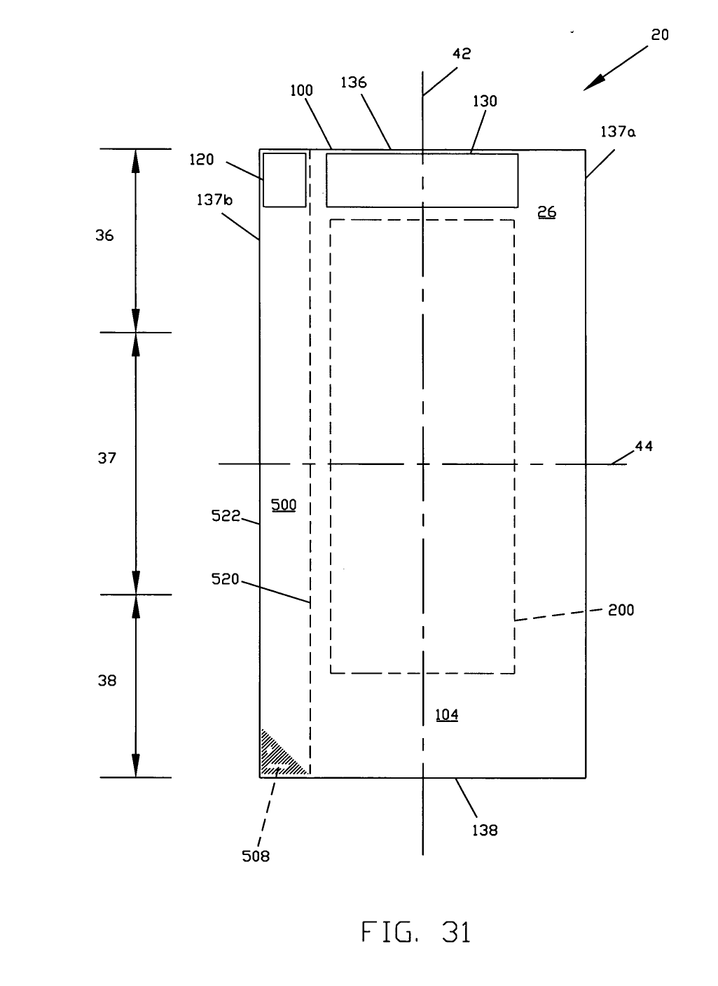

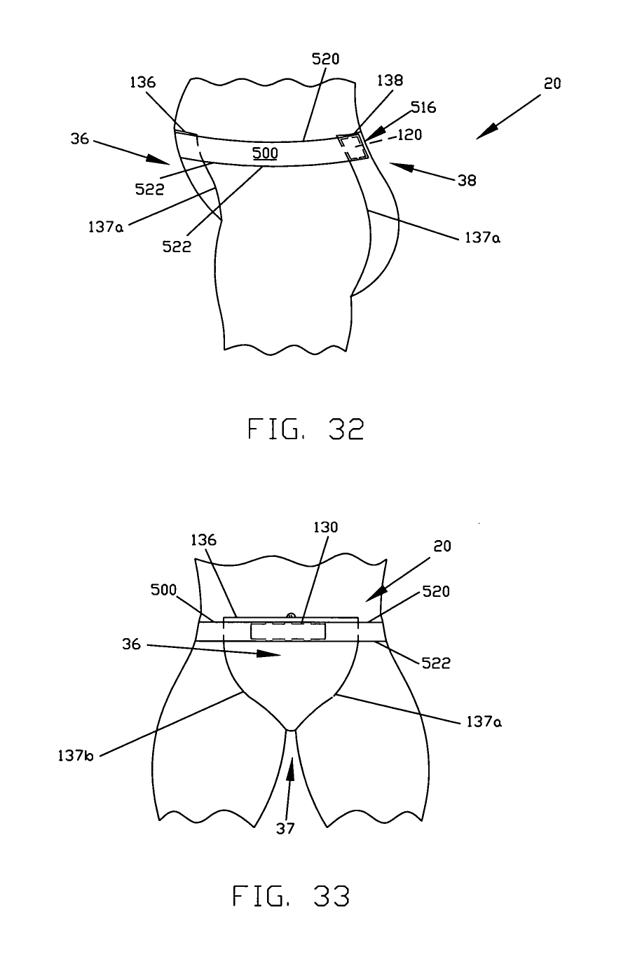

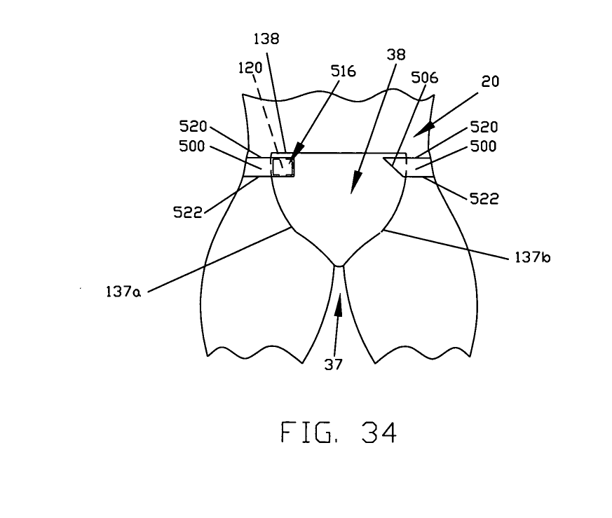

As another alternative, as shown in FIG. 31 through FIG. 34, the diaper 20 may have only a single deployable belt strip 500. When the diaper 20 is applied onto the wearer, such a “full length” belt strip 500 may be long enough to extend across the entirety of the opposing waist region and back to the starting waist region. In other words, a single full length belt strip 500 may be used to connect the waist regions at and/or adjacent to both of the side edges of the diaper 20. For example, as shown in FIG. 32, FIG. 33, and FIG. 34, the single belt strip 500 in FIG. 31 extends from the diagonal fold line 506 adjacent to the right side edge 137b in the back waist region 38 across the front waist region 36 and to the back waist region 38 such that its free end portion 516 lies adjacent to the left side edge 137a in the back waist region 38.

As shown in FIG. 5, FIG. 6, FIG. 14, and FIG. 30, two deployed belt strips 500 may be tied together in a knot 538 when they are long enough to make this practical. Alternatively, a fastener may be used to attach two deployed belt strips 500 together. Prior to fastening, the fastener may be disposed on either of the two belt strips 500. For example, in FIG. 26, the fastener 120a is used to attach the back left belt strip 500a to the front left belt strip 500c and the fastener 120b is similarly used to attach the back right belt strip 500b to the front right belt strip 500d. Alternatively, complementary fasteners may be disposed on matching belt strips 500, e.g., a hook patch may be disposed on one belt strip and a complementary loop patch may be disposed on another belt strip such that the two belt strips may be fastened together.

Alternatively, a fastener may be used to attach a deployed belt strip 500 to another portion of the diaper 20. Prior to fastening, the fastener may be disposed on the belt strip 500 or may be disposed on the other portion of the diaper 20 to which the belt strip 500 is to be fastened. For example, in FIG. 22 and FIG. 23, each fastener 120 is used to attach the respective belt strip 500 to the front waist region 36 of the diaper 20. As another example, in FIG. 32 and FIG. 34, the single fastener 120 is used to attach the single belt strip 500 to the back waist region 38. Alternatively, complementary fasteners may be used, e.g., a hook patch may be disposed on a belt strip and a complementary loop patch may be disposed on the other portion of the diaper 20 to which the belt strip is to be fastened.

The fastener 120 may be any type of fastening device suitable for the materials involved, for example an adhesive fastener, a cohesive fastener, a hook, a loop, a button, a patch of hooks, a patch of loops, etc. A fastener in the form of a patch of hooks that engage a nonwoven material may be suitable in some embodiments. The fastening of the belt strip 500 may become permanent once it is made, such that it cannot be undone without damage to the structural elements involved. Alternatively, the fastening of the belt strip 500 can be releasable and refastenable, such that it can be released for adjustment or for inspection of the interior of the diaper 20 and then refastened as before. The belt strip 500 may be fastened and/or tied at and/or adjacent to its free end portion 516. Alternatively or in addition, the belt strip 500 may be fastened and/or tied at one or more intermediate points between the diagonal fold line 506 and the free end portion 516.

When a deployed belt strip 500 is attached to a waist region by a fastener 120 or when two deployed belt strips 500 are tied or fastened together at a side of the body as in FIG. 30, both waist regions of the diaper 20 will be supported by the belt strips 500 that are attached to them. However, when two deployed belt strips 500 are tied together over a waist region to which they are not attached, as in FIG. 5 and in FIG. 14, or when a deployed belt strip 500 passes completely across a waist region to which it is not attached, as in FIG. 33, that waist region may tend to slide downward, i.e., toward the crotch region 37, relative to the belt strip 500, depending on the coefficients of static and dynamic friction between the waist region and the belt strip 500. In some embodiments, this inherent friction may be sufficient to prevent relative movement. Alternatively, it may be necessary and/or desirable to supplement such inherent friction in order to ensure that the waist region will not slide downward.

For example, in FIG. 1 through FIG. 5, in FIG. 10 through FIG. 14, and in FIG. 19, the belt strips 500 pass through laterally spaced belt loops 536, each of which is attached to the front waist region 36. Each belt loop 536 transfers force from the waist region to the belt strip 500 and thereby supports the front waist region 36 from the belt strip 500. The belt loops 536 may be attached to the waist region by any means suitable for the materials involved, including stitching, adhesive bonding, thermal bonding, stapling, and riveting, for example. For example, the belt loops 536 in the present figures are shown attached in attachment zones 537.

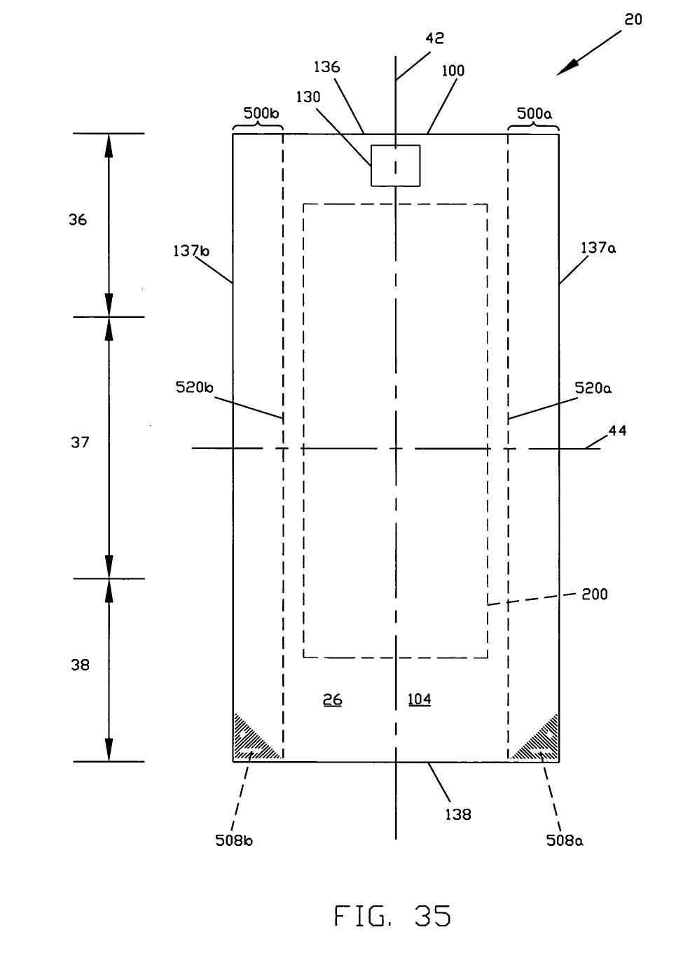

Optionally, as shown in FIG. 31 FIG. 33, and FIG. 35, an additional fastener 130 similar to any of the fasteners 120 described above may be disposed on the front waist region 36, where it will be overlapped by a belt strip 500, in order to transfer force from the waist region to the belt strip 500 and thereby support the waist region from the belt strip 500. Such a fastener may be disposed on the belt strip 500, instead of on the chassis 100 as shown. A suitable fastener may be relatively wide as shown in FIG. 31 and FIG. 33 or relatively narrow as shown in FIG. 35 and may have any shape, such as the rectangular shape shown in these figures. As an alternative to a fastener, a friction patch having a relatively high coefficient of static friction may be used. The fastener or friction patch may be disposed such that the belt strip may be overlapped exteriorly of the waist region, as shown in the figures. Alternatively, the fastener or friction patch may be disposed such that the belt strip lies against the body of the wearer and the waist region is overlapped exteriorly of the belt strip.

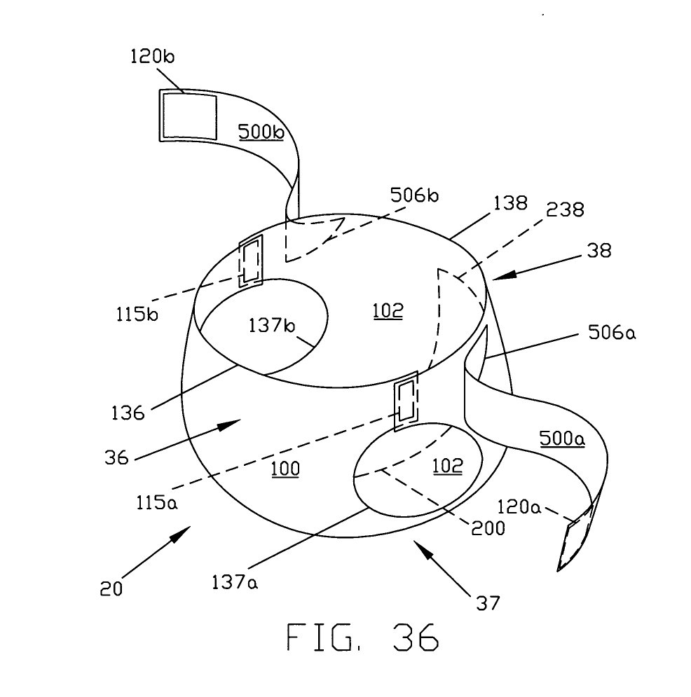

As shown in FIG. 36, an exemplary diaper 20 having exteriorly disposed belts strips 500 may have side seams 115 at which the front and back waist regions 36 and 38 are non-releasably attached together adjacent to the respective side edges 137 and thereby have the form of pants. In such an embodiment, the belt strips 500 can be used to set and/or adjust the tightness around the waist of the wearer.

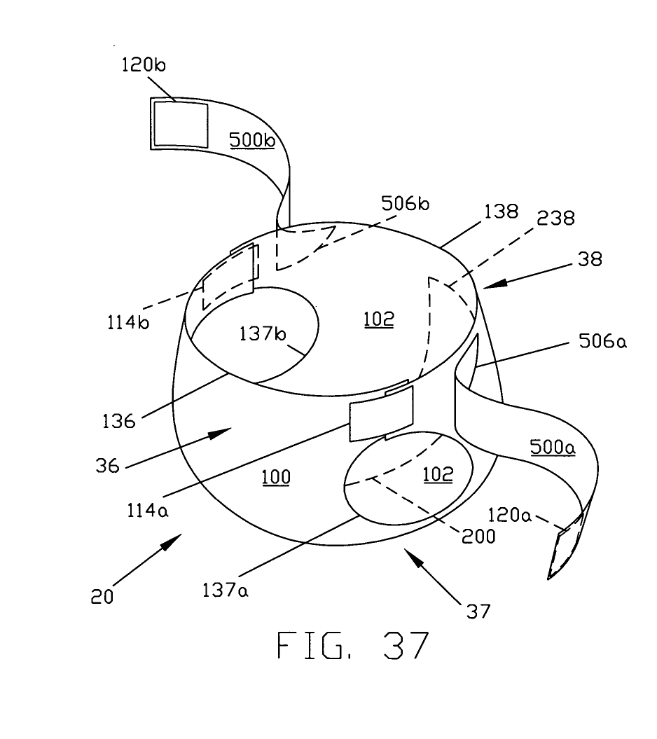

Similarly, as shown in FIG. 37, an exemplary diaper 20 having exteriorly disposed belts strips 500 may have side fasteners 114 by which the front and back waist regions 36 and 38 are attached together adjacent to the respective side edges 137. The side fasteners 114 may be releasable and refastenable, thereby allowing for easy inspection of the interior of the diaper 20 while it is being worn and subsequent refastening when it is not necessary to change the diaper. The diaper 20 may be provided to the user with the side fasteners 114 already fastened or in an unfastened condition.

A belt strip 500 may be formed contiguously with another structural element of the diaper 20. At least one edge of such a contiguous belt strip 500 is defined by a frangible separation line along which the belt strip 500 can be partially detached for use. Such a frangible separation line may be formed in a layer or a laminate of layers by perforation, by the formation of a brittle area or areas at which the material will preferentially fracture when stressed, by the formation of a weaker area or areas at which the material will preferentially tear when stressed, by the formation of a friable area or areas at which the material will preferentially crumble when stressed and/or bent, or by any other method of providing frangibility that is suitable for the materials involved.

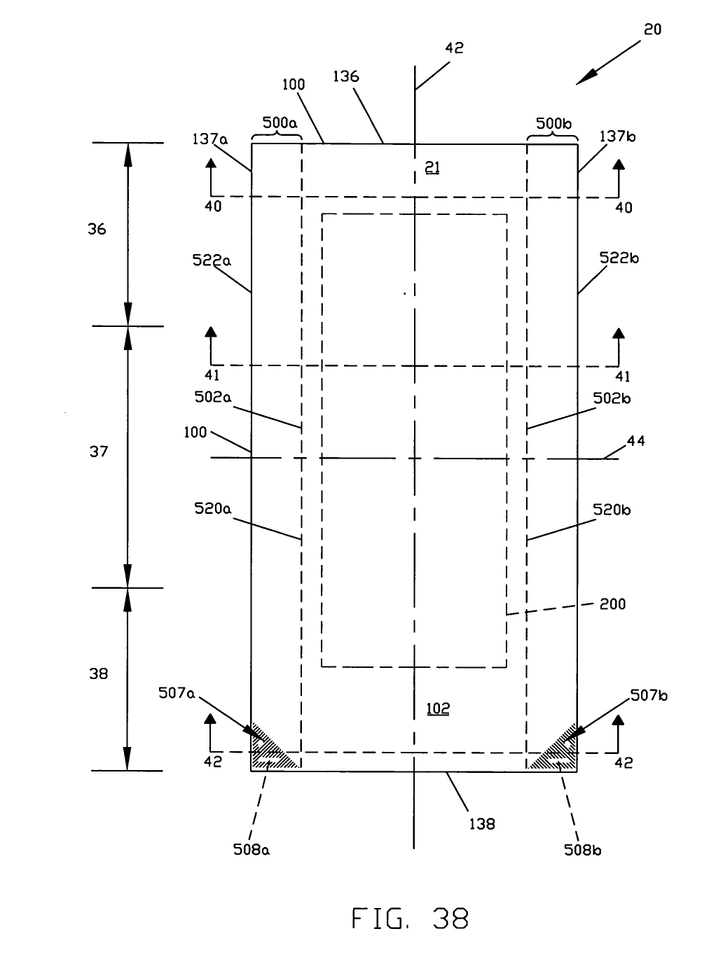

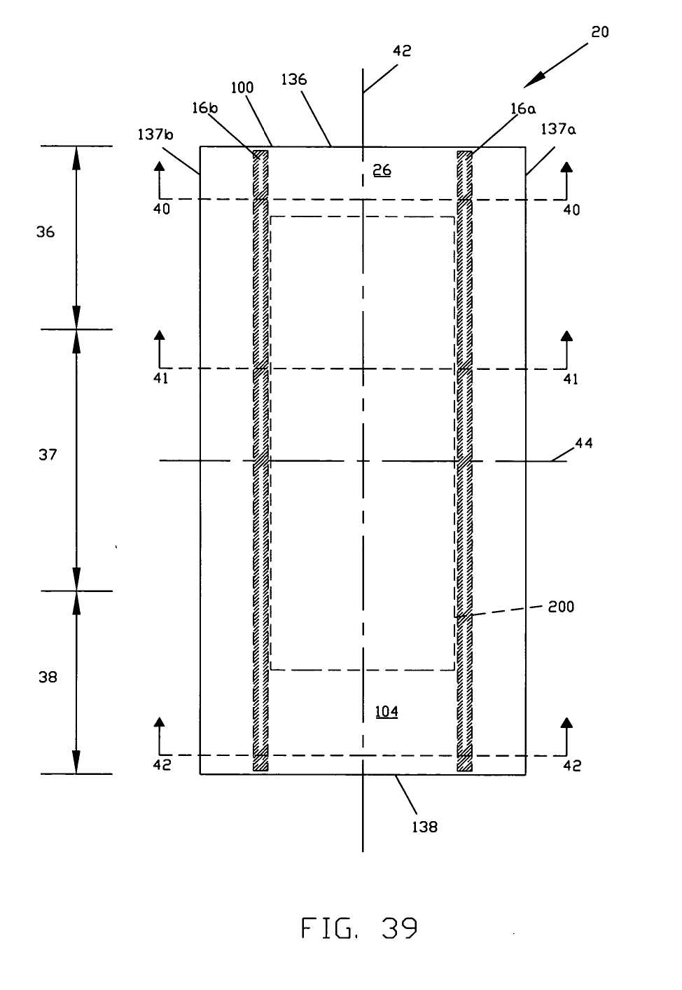

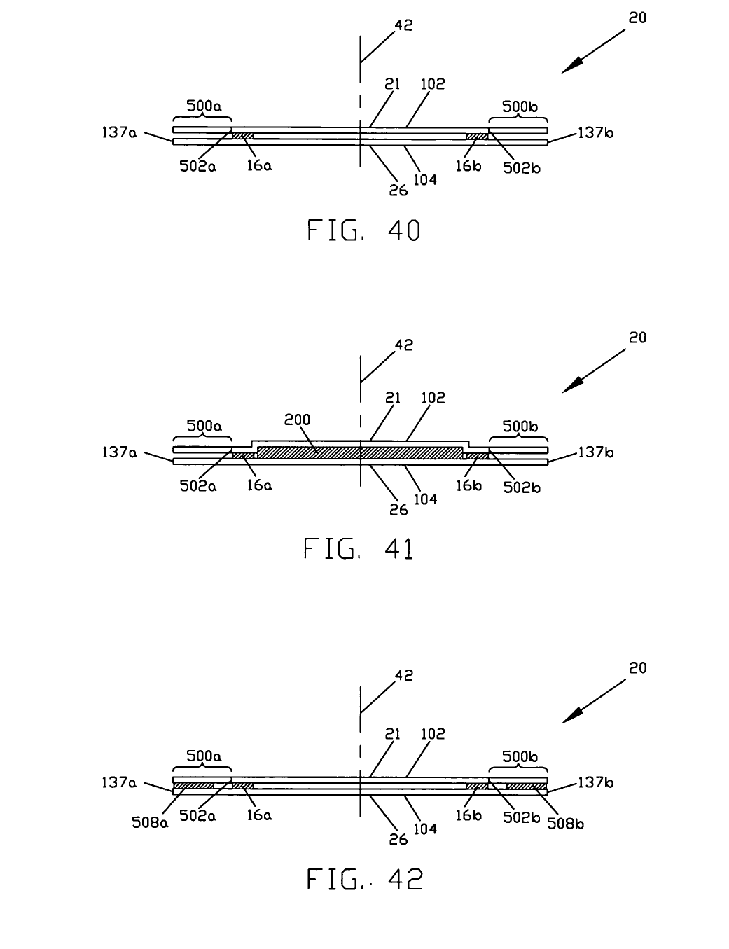

For example, the diaper 20 in FIG. 38 through FIG. 42 has a conventional diaper structure, in which a liquid permeable topsheet 21 defines its interior surface 102, a liquid impermeable backsheet 26 defines its exterior surface 104, and an absorbent assembly 200 is sandwiched between the topsheet and the backsheet. In this example, each interiorly disposed belt strip 500 is formed from the same layer as the topsheet 21 by two laterally inboard frangible separation lines 502. This configuration may be used, for example, in order to ensure that the same soft material used for the topsheet 21 contacts the wearer's skin wherever the belt strip 500 lies on the body when deployed for use.

As can be readily understood by reference to the preceding description of various configurations of belt strips, in this example, each frangible separation line 502 corresponds to the first edge 520 of the respective belt strip 500 and each belt strip 500 can be deployed for use by being partially detached along its frangible separation line 502 and then folded outward along its diagonal fold line (not shown), which is defined during the deployment by its attachment zone 508. Also shown in this example is an optional longitudinally extending attachment zone 16 where the topsheet 21 is attached to the backsheet 26 generally parallel and adjacent to each of the frangible separation lines 502. These attachment zones 16 may be included if desired for structural stability and/or strength, but may be excluded if desired to minimize cost. If included, these attachment zones 16 may be continuous or may be intermittent. In addition, if desired for reasons related to handling, packaging, or appearance prior to deployment of the belt strips 500, each belt strip 500 may be releasably attached to another layer with which it is in face-to-face contact. For example, each belt strip 500 may be releasably attached to the backsheet 26 adjacent to its second edge 522 and/or adjacent to the front edge 36 of the diaper 20.

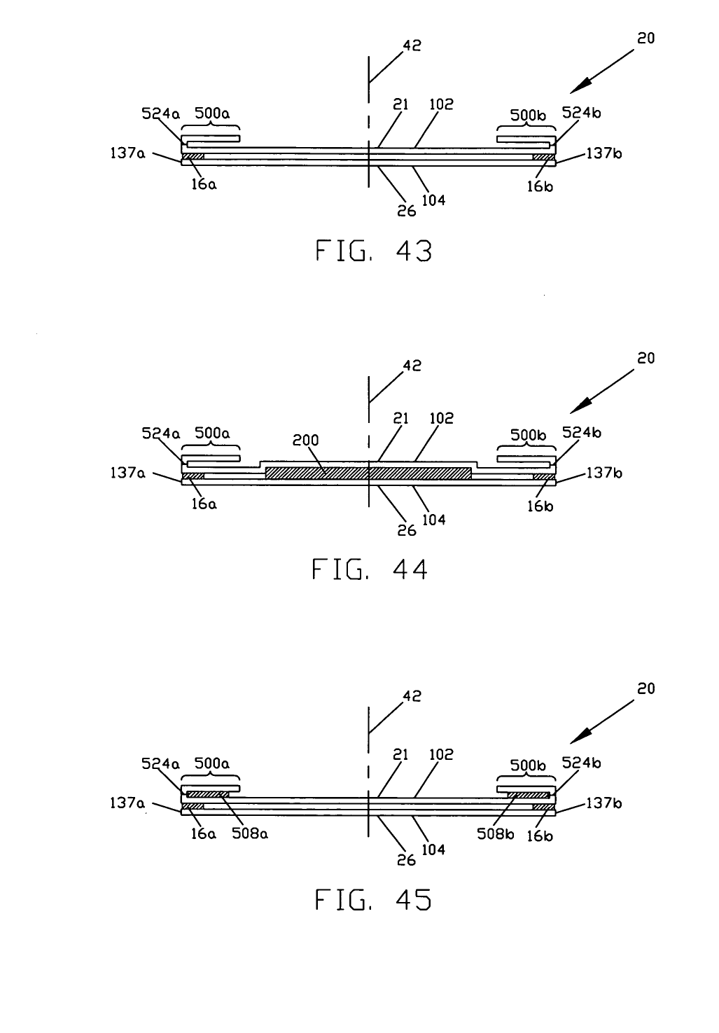

Another example is shown in FIG. 43 through FIG. 45. The topsheet 21 in this example is folded laterally inward at or adjacent to the side edges 137 of the diaper 20 and the interiorly disposed belt strips 500 are again formed from the same layer as the topsheet 21. However, in this example, the two laterally outboard frangible separation lines 524 correspond to the second edges 522 of the belt strips 500. Also in this example, in order for the optional releasable attachment zones 16 to again be located adjacent to the frangible separation lines, the optional releasable attachment zones 16 are located adjacent to the side edges 137.

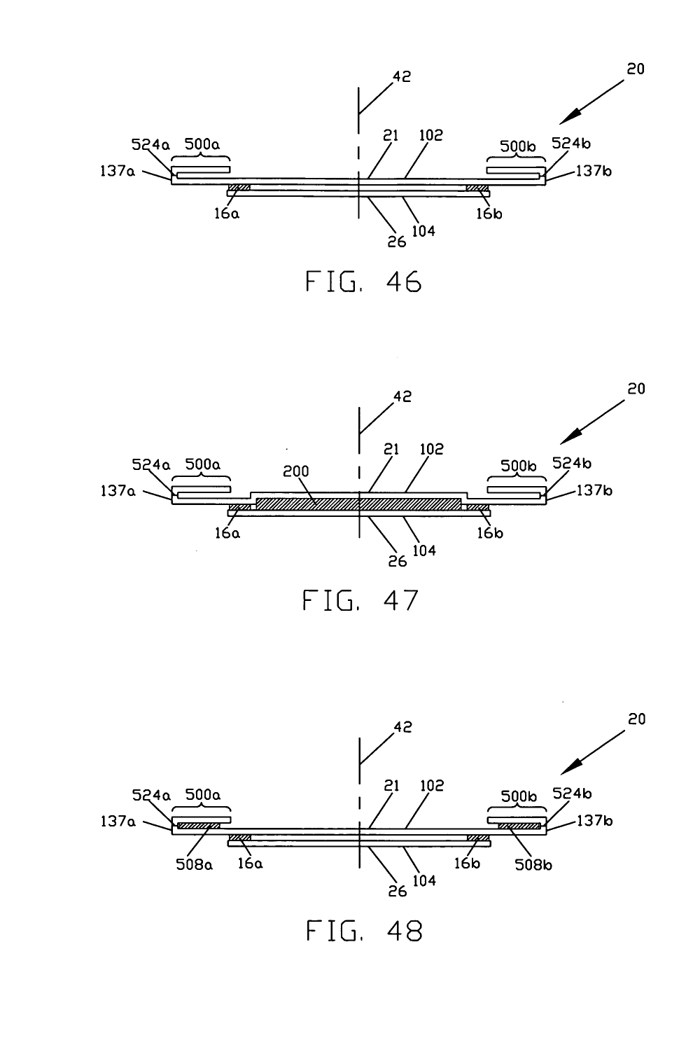

In the example shown in FIG. 46 through FIG. 48, the topsheet 21 is similarly folded laterally inward at or adjacent to the side edges 137 of the diaper 20 and the interiorly disposed belt strips 500 are again formed from the same layer as the topsheet 21. However, in this example, the backsheet 26 is laterally narrower than the topsheet 21 and so the optional releasable attachment zones 16 are located relatively farther laterally inboard of the side edges 137 than in the preceding example. Such a configuration might be chosen in order to minimize the amount of backsheet material used and thereby minimize the cost of the diaper 20.

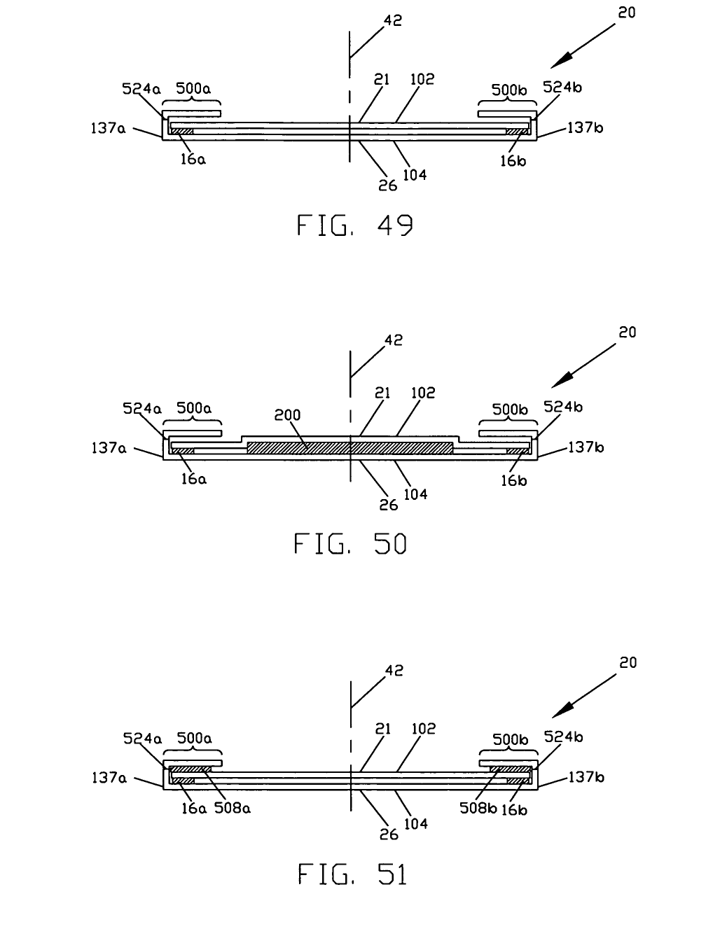

In the exemplary diaper 20 shown in FIG. 49 through FIG. 51, the backsheet 26 is folded laterally inward to wrap the topsheet 21 at or adjacent to the side edges 137 of the diaper 20 and the interiorly disposed belt strips 500 are formed from the same layer as the backsheet 26. A configuration in which the belt strips 500 are formed from the same material as the backsheet 26 may be used when, for example, the backsheet material has a relatively greater tensile strength than the topsheet material and the greater strength is required in the belt strips 500.

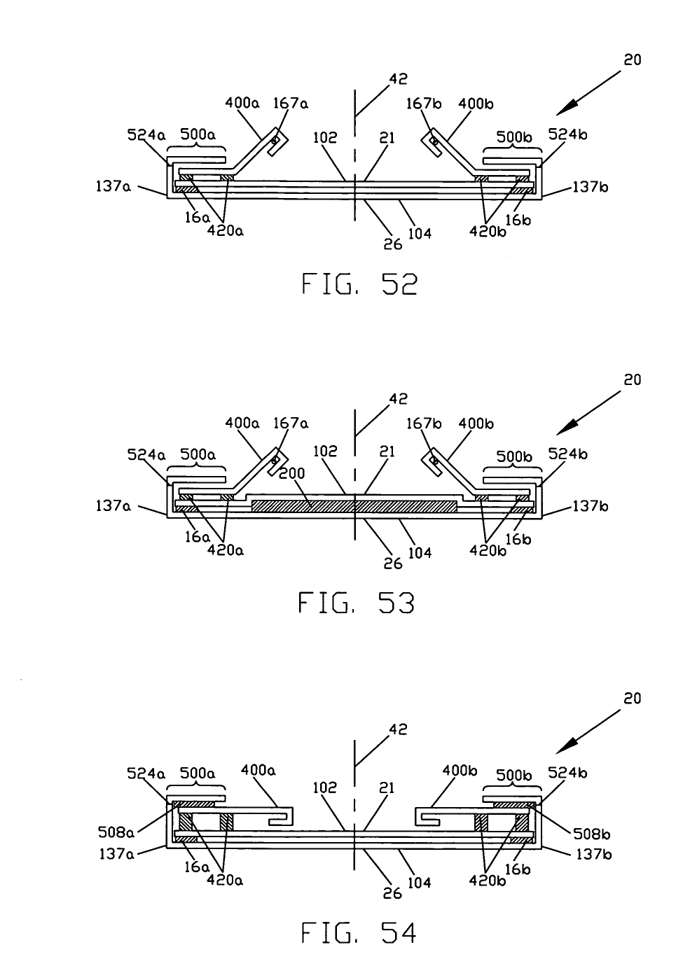

The next example shown in FIG. 52 through FIG. 54 is similar in its basic structure, but in this example, barrier cuffs 400 are shown attached to the topsheet 21 at attachment zones 420. Each barrier cuff 400 incorporates an elastic member 167 that contracts and raises the barrier cuff 400 to form a barrier to the flow of bodily waste in a direction laterally away from the longitudinal axis 42, as is well-known in the art. In such an embodiment, the optional attachment of the topsheet 21 to the backsheet 26 at the attachment zones 16 may be useful in order to provide additional anchoring of the barrier cuffs 400 adjacent to the side edges 137.

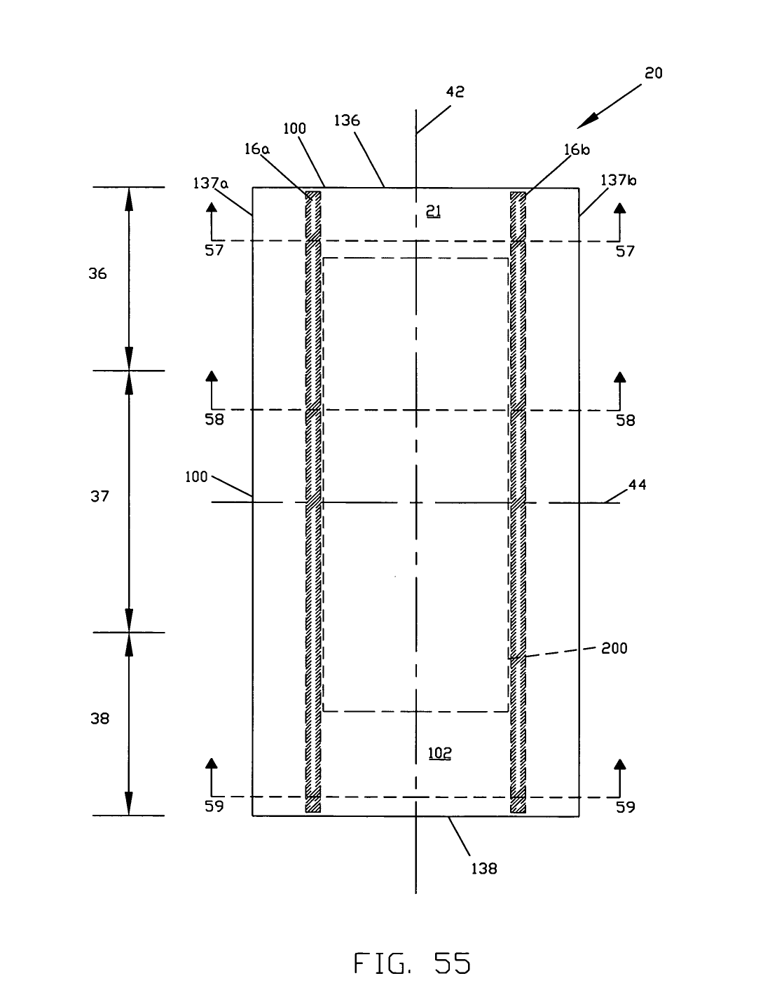

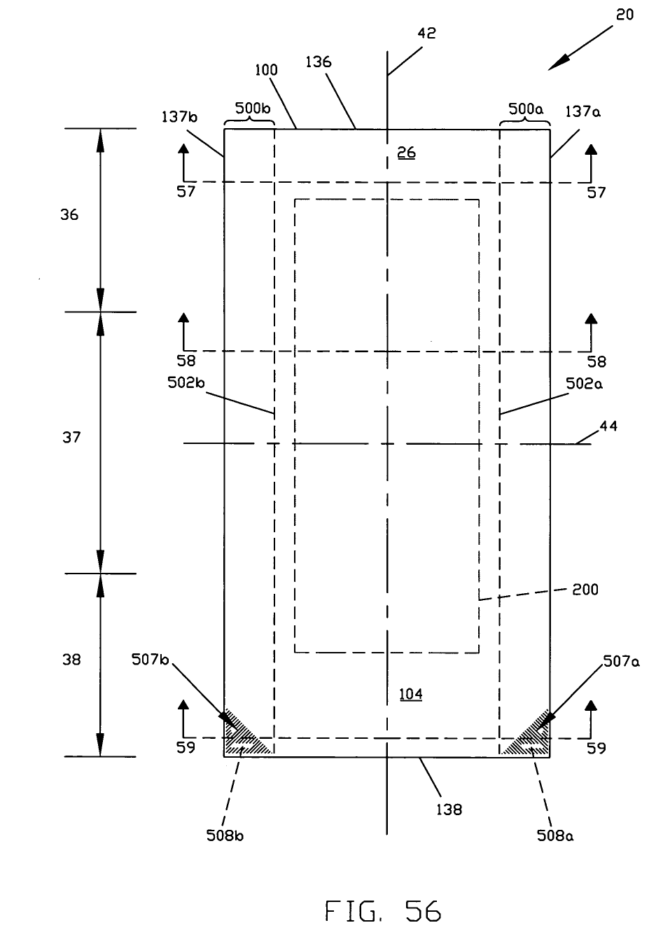

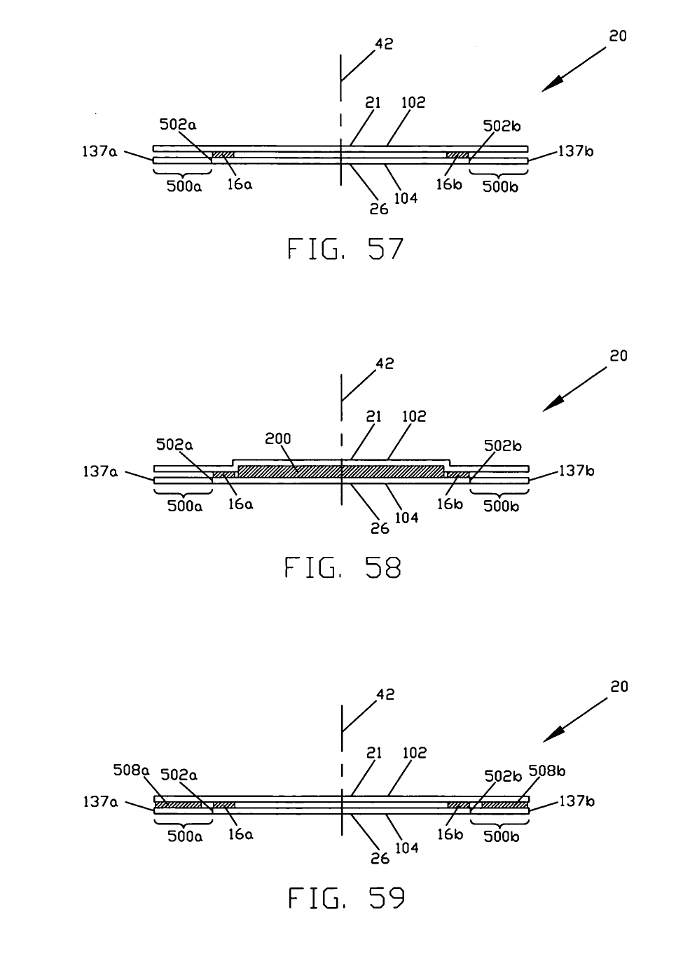

Exteriorly disposed belt strips 500 may also be formed contiguously with another structural element of the diaper 20. For example, the diaper 20 in FIG. 55 through FIG. 59 again has a conventional diaper structure. In this example, two exteriorly disposed belt strips 500 are formed from the same layer as the backsheet 26 by two laterally inboard frangible separation lines 502. Each frangible separation line 502 again corresponds to the first edge 520 of the respective belt strip 500 and each belt strip 500 can again be deployed for use by being partially detached along its frangible separation line 502 and then being folded laterally outward.

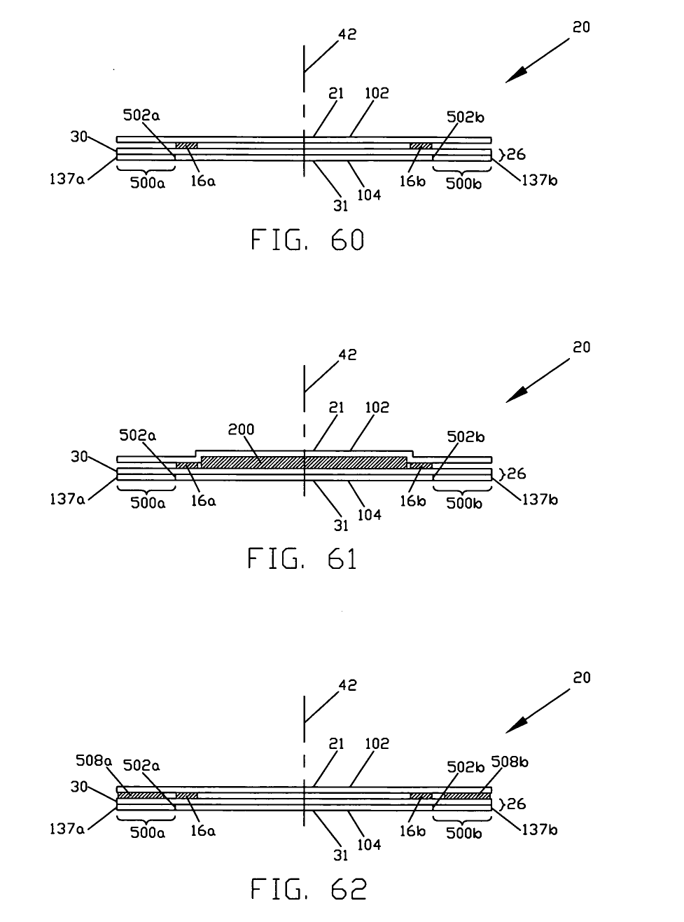

In the example shown in FIG. 60 through FIG. 62, the backsheet 26 is a laminate of layers 30 and 31. In this example, the frangible separation lines 502 extend through only the lower layer 31 of the backsheet 26. Thus, when the exteriorly disposed belt strips 500 are deployed for use, the upper layer 30 of the laminate backsheet 26 remains intact. Such a configuration may be used, for example, when the lower layer 31 of the laminate backsheet 26 is a nonwoven material and it is desired to use a nonwoven material for the belt strips 500. Alternatively, the frangible separation lines may extend through both layers of such a laminate.

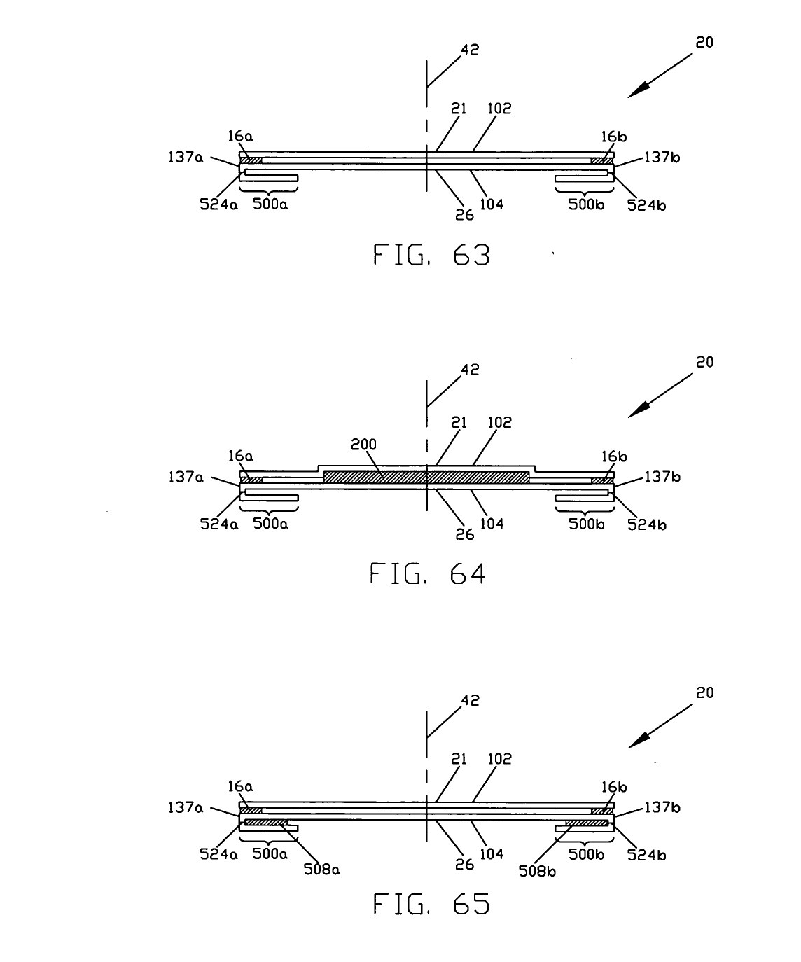

The backsheet 26 in the example shown in FIG. 63 through FIG. 65 is folded laterally inward at or adjacent to the side edges 137 of the diaper 20 and the exteriorly disposed belt strips 500 are again formed from the same layer as the backsheet 26 by two laterally outboard frangible separation lines 524 located adjacent to the side edges 137 of the diaper 20.

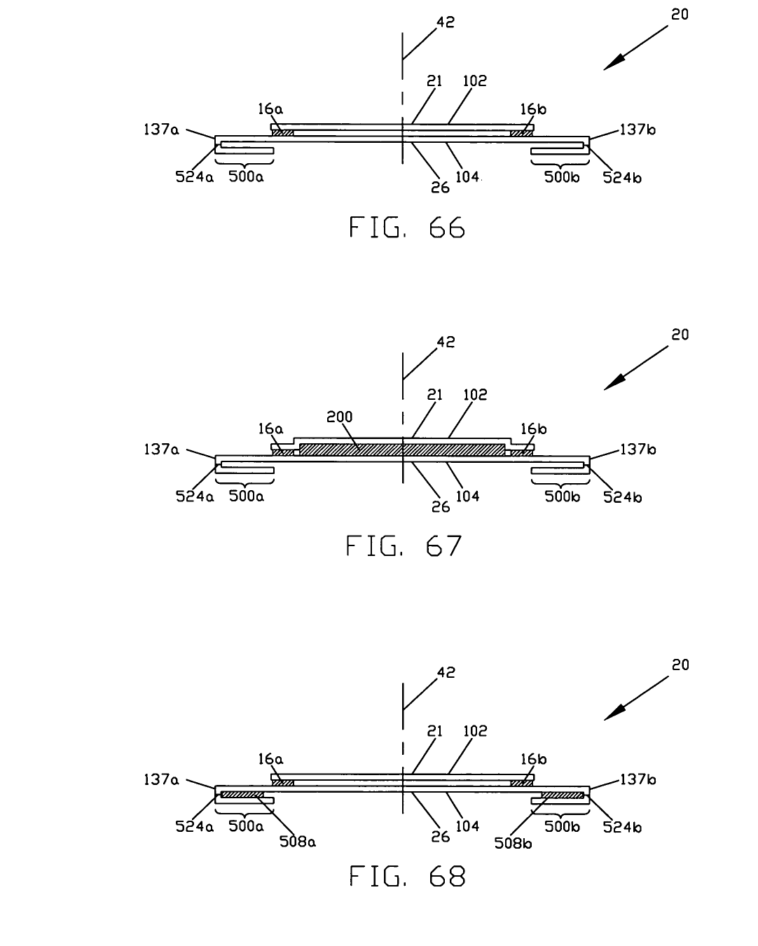

In the example shown in FIG. 66 through FIG. 68, the backsheet 26 is similarly folded laterally inward at or adjacent to the side edges 137 of the diaper 20 and the exteriorly disposed belt strips 500 are again formed from the same layer as the backsheet 26. However, in this example, the topsheet 21 is laterally narrower than the backsheet 26 and so the optional releasable attachment zones 16 are located relatively farther laterally inboard of the side edges 137 than in the preceding example. Such a configuration might be chosen in order to minimize the amount of topsheet material used and thereby minimize the cost of the diaper 20.

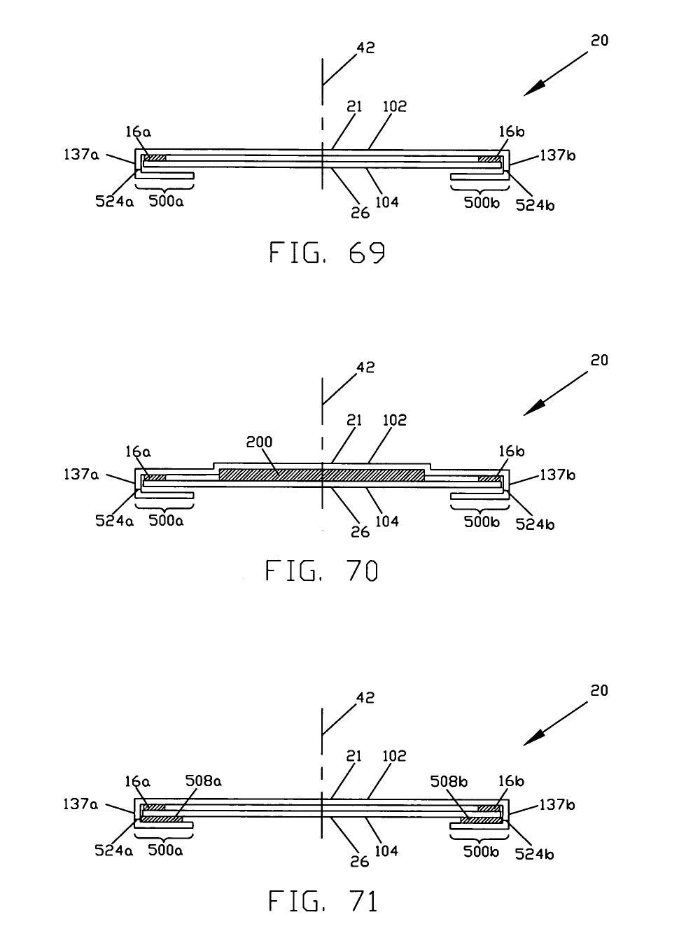

In the exemplary diaper 20 shown in FIG. 69 through FIG. 71, the topsheet 21 is folded laterally inward to wrap the backsheet 26 at or adjacent to the side edges 137 of the diaper 20 and the exteriorly disposed belt strips 500 are formed from the same layer as the topsheet 21.

Alternatively, a belt strip 500 may be formed discretely rather than contiguously with another element of the diaper 20. A configuration in which the belt strip is discretely formed may be chosen, for example, when it is desired to use a particular material for the belt strip that is different from either the topsheet material or the backsheet material. For simplicity in this description, the discretely formed belt strips in the following examples are shown and described in exterior dispositions but, as with contiguously formed belt strips, discretely formed belt strips may be disposed either interiorly or exteriorly.

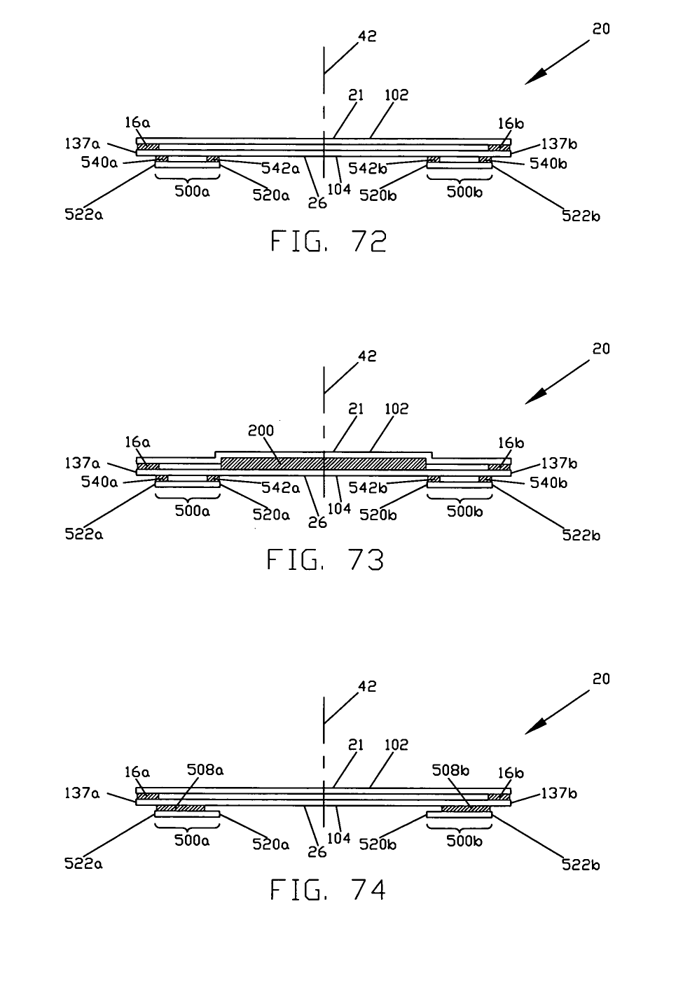

For example, in the diaper 20 shown in FIG. 72 through FIG. 74, the exteriorly disposed belt strips 500 are releasably attached to the backsheet 26 at longitudinally extending laterally inboard attachment zones 542 and longitudinally extending laterally outboard attachment zones 540, as well as at the attachment zones 508 located opposite their free end portions. The belt strips 500 in this example are laterally spaced apart similarly to the previously described contiguously formed belt strips 500. These belt strips 500 can be deployed for use by detaching them at the attachment zones 540 and 542 and folding them laterally outward, as in the previous examples. It may be desirable to choose such a configuration in which a belt strip 500 is releasable for deployment at an attachment zone in order to minimize the number of frangible separation lines that must be formed during the manufacture of the diaper 20. Alternatively, the choice of materials may make the choice of frangible separation lines instead of releasable attachments relatively more desirable.

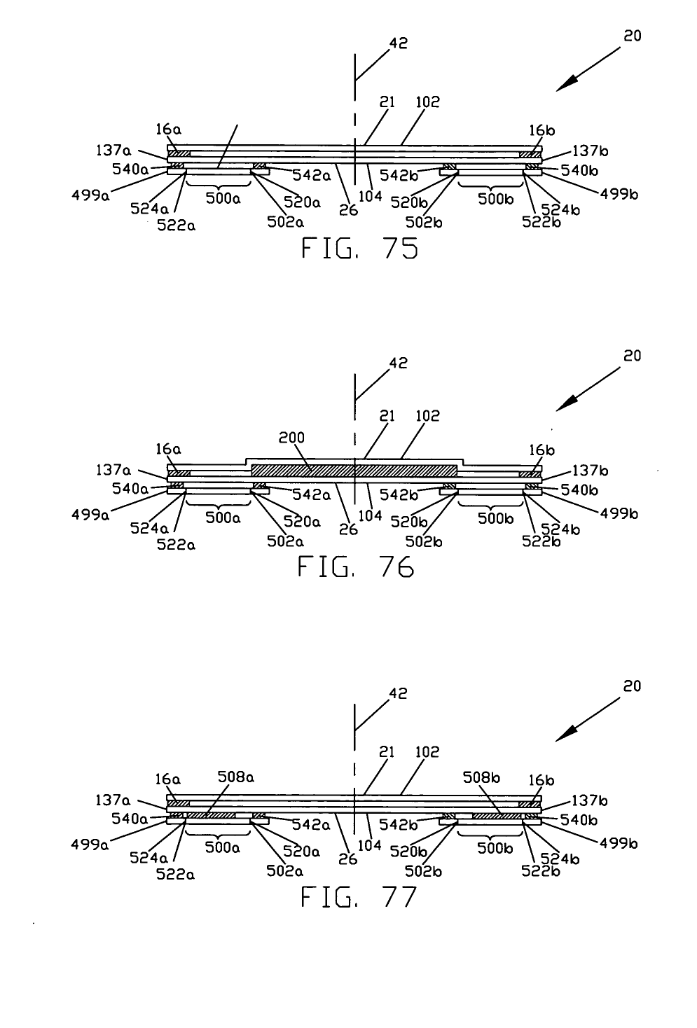

In the next example shown in FIG. 75 through FIG. 77, two discrete strips 499 are attached to the backsheet 26 at longitudinally extending laterally inboard attachment zones 542 and longitudinally extending laterally outboard attachment zones 540, but these attachments are not releasable as in the preceding example. Instead, an exteriorly disposed belt strip 500 is defined in each of the discrete strips 499 by a laterally inboard frangible separation line 502 and a laterally outboard frangible separation line 524, at which the belt strip 500 can be detached for deployment. As in the previous examples, the laterally inboard frangible separation line 502 corresponds to the first edge 520 of the belt strip 500 and the laterally outboard frangible separation line 524 corresponds to the second edge 522 of the belt strip 500. Such a configuration in which a discrete strip 499 is not releasable at its attachment to the backsheet 26 may be chosen, for example, when it is desired to avoid the exposure of an adhesive used in the attachment zone 540 after deployment of the belt strip 500.

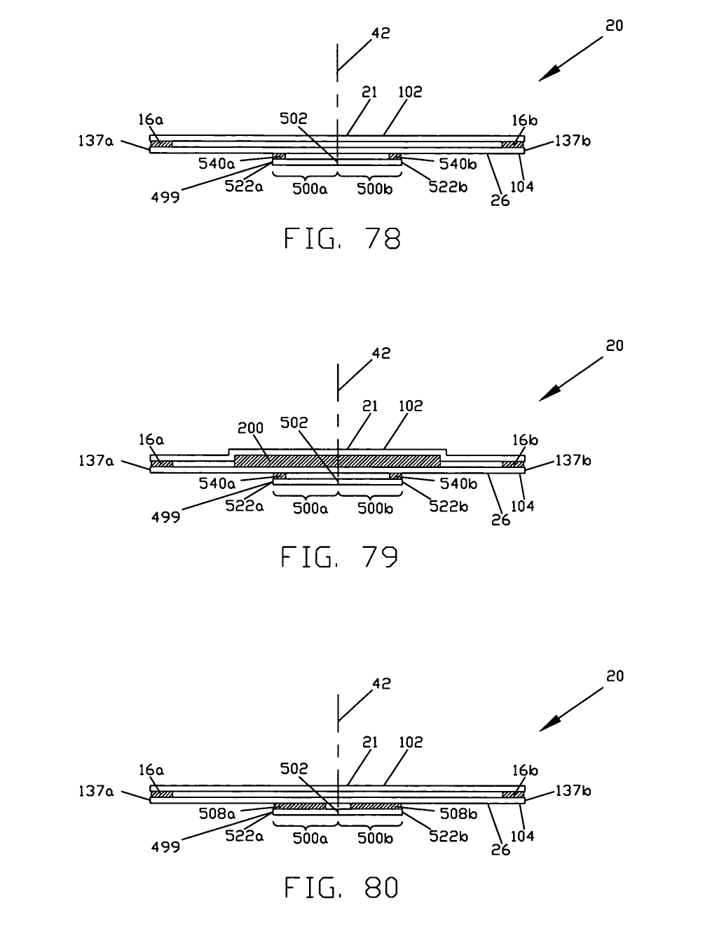

A single discrete strip 499 is attached to the backsheet 26 of the diaper 20 shown in FIG. 78 through FIG. 80 at longitudinally extending laterally spaced attachment zones 540. A single common frangible separation line 502 located between the attachment zones 540 in this discrete strip 499 defines the two laterally abutted exteriorly disposed belt strips 500. In this example, the belt strips 500 are releasably attached at the attachment zones 540 such that their deployment can be effected by detaching them there and at the frangible separation line 502. Such a laterally abutted arrangement of the belt strips 500 may be desirable, for example, in order to concentrate the forces exerted by deployed belt strips on a relatively smaller area of the diaper 20 than is the case when the belt strips are laterally spaced apart. Also, in some embodiments, such laaterally abutted exteriorly disposed belt strips may provide a relatively more finished appearance to the diaper 20 when it worn with the belt strips deployed around the waist of the wearer, due to the relatively greater extent of encirclement of the waist that is achievable with this configuration. Such a configuration of the belt strips may also be desirable in order to relatively simplify the process for manufacturing the diaper 20 by requiring only a single discrete strip 499 for the formation of two belt strips 500.

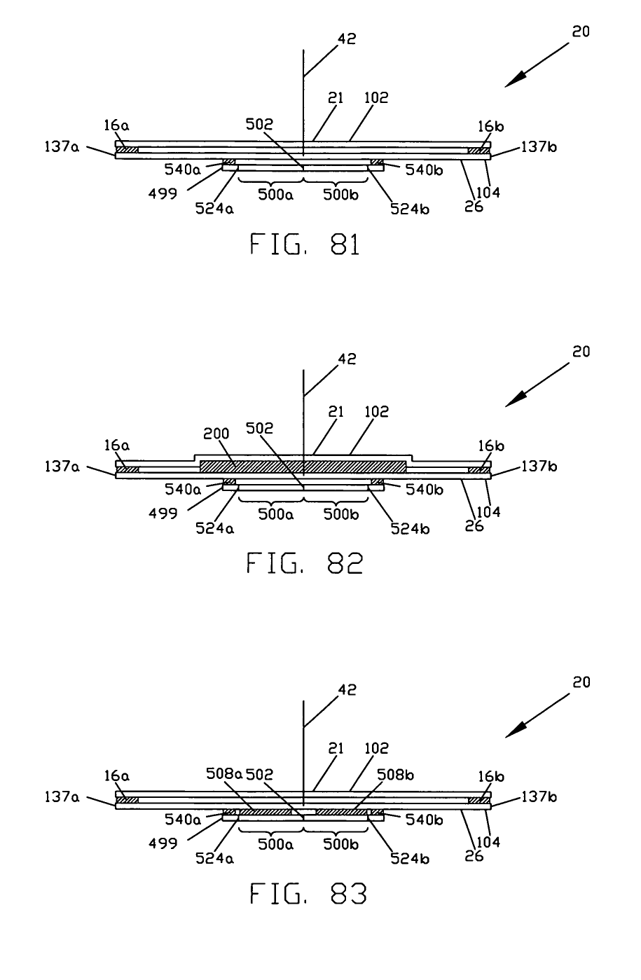

In the next example shown in FIG. 81 through FIG. 83, a single discrete strip 499 is similarly attached to the backsheet 26 at longitudinally extending laterally spaced attachment zones 540, but these attachments are not releasable as in the preceding example. Instead, a pair of laterally abutted exteriorly disposed belt strips 500 is defined in the single discrete strip 499 by a single common frangible separation line 502 and two laterally outboard frangible separation lines 524, at which the belt strips 500 can be detached for deployment. As in the previous examples, the common frangible separation line 502 corresponds to the first edges 520 of the belt strips 500 and the laterally outboard frangible separation lines 524 correspond to the second edges 522 of the respective belt strips 500.

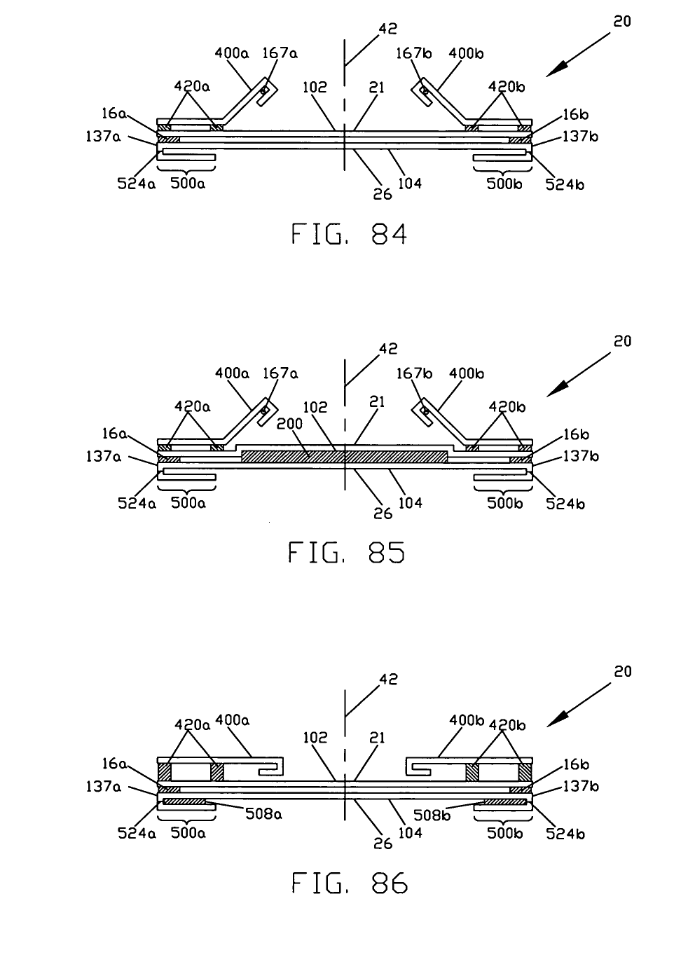

In the next example shown in FIG. 84 through FIG. 86, the backsheet 26 is folded laterally inward at or adjacent to the side edges 137 of the diaper 20 and the exteriorly disposed belt strips 500 are formed from the same layer as the backsheet 26, as in the example shown in FIG. 63 through FIG. 65. However, in this example, barrier cuffs 400 are shown attached to the topsheet 21 at attachment zones 420 and the optional attachment of the topsheet 21 to the backsheet 26 at the attachment zones 16 is also shown.

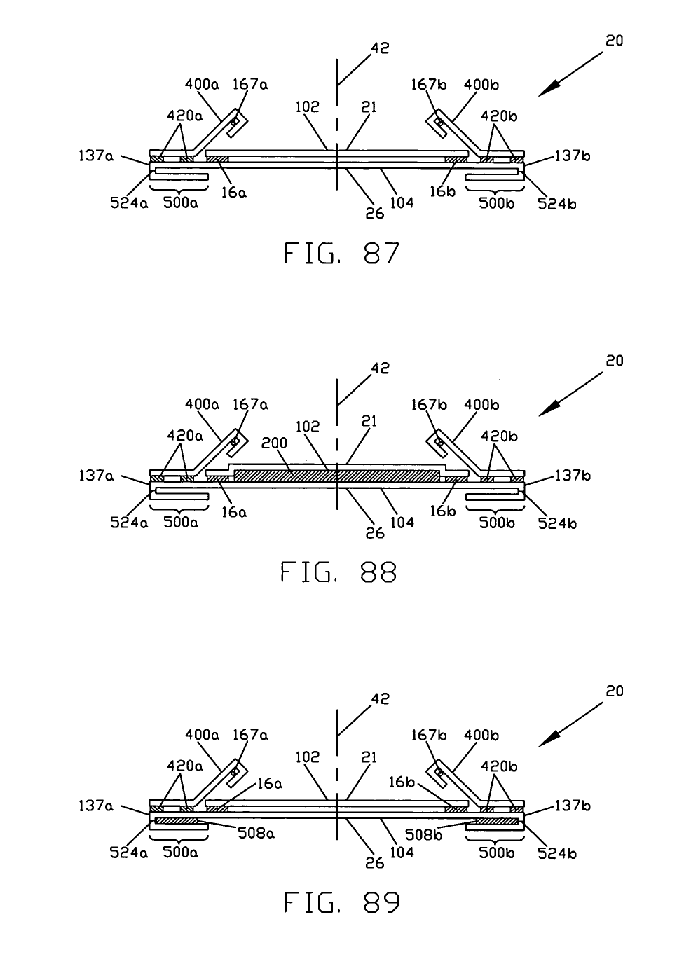

In the example shown in FIG. 87 through FIG. 89, the backsheet 26 is similarly folded laterally inward at or adjacent to the side edges 137 of the diaper 20 and the exteriorly disposed belt strips 500 are again formed from the same layer as the backsheet 26. However, in this example, the topsheet 21 is laterally narrower than the backsheet 26 and so the optional releasable attachment zones 16 are located relatively farther laterally inboard of the side edges 137 than in the preceding example.

The preceding examples are provided in order to convey to persons of skill in the art that the deployable belt strips of the present invention can be provided in a variety of configurations. The above examples are not exhaustive, i.e., variations in addition to these are foreseen. For example, each of the mentioned layers, e.g., the topsheet 21, the backsheet 26, the belt strips 500, etc., may be formed of two or more members and thus may be laminates and/or composites of such members. As another example, each of the mentioned layers may be doubled by folding such that, for example, a belt strip 500 may be doubled and have one edge defined by a fold. The intent is to convey the concept of the present invention, i.e., a diaper incorporating deployable belt strips, while avoiding unnecessary length and complexity in this description. This voluntary characterization of the present invention is expressly not intended to constitute a surrender of any potential scope of any patentable claim(s).

The disclosures of all patents, patent applications and any patents which issue thereon, as well as any corresponding published foreign patent applications, and all publications listed and/or referenced in this description, are hereby incorporated in their entireties herein by reference. It is expressly not admitted that any of the documents or any combination of the documents incorporated herein by reference teaches or discloses the present invention.

What is claimed is:

1. A disposable diaper comprising:
a chassis comprising a liquid permeable topsheet and a liquid impermeable backsheet and having longitudinally opposing front and back waist regions having waist edges, laterally opposing side edges connecting the waist edges, and a crotch region between the waist regions;
an absorbent assembly sandwiched between the topsheet and the backsheet; and
at least one belt strip having a fixed end portion, an opposing free end portion, a first edge, and a second edge disposed laterally outboard of the first edge, the first edge and the second edge connecting the end portions, the belt strip being attached in the fixed end portion in one of the waist regions and additionally being attached to the chassis along at least a portion of one of the first edge and the second edge,
the belt strip being deployed by being detached from the chassis except at its fixed end portion and folded laterally outward at a diagonal fold line having opposing end points such that the first edge extends laterally outward from one end point and the second edge extends laterally outward from the opposing end point.

2. The disposable diaper of claim 1 wherein, prior to its deployment, the belt strip extends to the waist edge of the waist region opposing its fixed end portion.

3. The disposable diaper of claim 1 wherein, prior to its deployment, the belt strip extends only from the waist region in which its fixed end portion is disposed into the crotch region.

4. The disposable diaper of claim 1 having only a single belt strip extending when deployed from the waist region where its fixed end portion is disposed to and laterally across the opposing waist region and further to the waist region where its fixed end portion is disposed and thereby connecting the waist regions at both of the side edges of the chassis.

5. The disposable diaper of claim 1 having two laterally opposing belt strips.

6. The disposable diaper of claim 5 wherein the belt strips are laterally spaced apart.

7. The disposable diaper of claim 5 wherein the belt strips are laterally abutted.

8. The disposable diaper of claim 5 wherein, after their deployment, the belt strips are tied together exteriorly of the waist region opposing their fixed end portions.

9. The disposable diaper of claim 5 wherein, after their deployment, the belt strips are attached by a fastener to the waist region opposing their fixed end portions.

10. The disposable diaper of claim 1 having two laterally opposing belt strips having their fixed end portions disposed in the front waist region and two laterally opposing belt strips having their fixed end portions disposed in the back waist region.

11. The disposable diaper of claim 10 wherein, after their deployment, two of the belt strips are tied together adjacent to one of the side edges and the other two of the belt strips are tied together adjacent to the opposing side edge.

12. The disposable diaper of claim 10 wherein, after their deployment, two of the belt strips are attached together by a fastener adjacent to one of the side edges and the other two of the belt strips are attached together by another fastener adjacent to the opposing side edge.

13. The disposable diaper of claim 1 wherein the belt strip is formed contiguously with another structural element of the disposable diaper.

14. The disposable diaper of claim 13 wherein the belt strip is detachable from the other structural element along a frangible separation line.

15. The disposable diaper of claim 1 wherein the belt strip is formed from a discrete strip attached to the chassis.

16. The disposable diaper of claim 1 wherein the fixed end portion is attached in an attachment zone extending longitudinally and laterally outward from the diagonal fold line.

17. A disposable diaper comprising:
a chassis comprising a liquid permeable topsheet and a liquid impermeable backsheet and having longitudinally opposing front and back waist regions having waist edges, laterally opposing side edges connecting the waist edges, and a crotch region between the waist regions;
an absorbent assembly sandwiched between the topsheet and the backsheet; and
at least one exteriorly disposed belt strip having a fixed end portion disposed in one of the waist regions, an opposing free end portion, a first edge and a second edge, the first edge and the second edge connecting the end portions,
the belt strip being folded laterally outward at a diagonal fold line such that the first edge extends laterally outward from a laterally proximal end point of the diagonal fold line and the second edge extends laterally outward from a laterally distal end point of the diagonal fold line.

18. The disposable diaper of claim 17 wherein the belt strip is formed contiguously with the backsheet.

19. The disposable diaper of claim 17 wherein the belt strip is formed from a discrete strip attached to the backsheet.

20. A disposable diaper comprising:
a chassis comprising a liquid permeable topsheet and a liquid impermeable backsheet and having longitudinally opposing front and back waist regions having waist edges, laterally opposing side edges connecting the waist edges, and a crotch region between the waist regions;
an absorbent assembly sandwiched between the topsheet and the backsheet; and
at least one interiorly disposed belt strip having a fixed end portion disposed in one of the waist regions, an opposing free end portion, a first edge and a second edge, the first edge and the second edge connecting the end portions,
the belt strip being folded laterally outward at a diagonal fold line such that the first edge extends laterally outward from a laterally proximal end point of the diagonal fold line and the second edge extends laterally outward from a laterally distal end point of the diagonal fold line.
